(19) United States
(12) Patent Application Publication
(10) Pub. No.: US 2025/0114074 A1
(43) Pub. Date: Apr. 10, 2025

(54) ULTRASOUND DIAGNOSTIC APPARATUS, IMAGE PROCESSING APPARATUS, MEDICAL INFORMATION-PROCESSING APPARATUS, ULTRASOUND DIAGNOSTIC METHOD, AND NON-TRANSITORY COMPUTER-READABLE RECORDING MEDIUM

(71) Applicant: CANON MEDICAL SYSTEMS CORPORATION, Tochigi (JP)

(72) Inventors: Masashi USUMURA, Nasushiobara (JP); Hiroki TAKAHASHI, Nasushiobara (JP)

(73) Assignee: CANON MEDICAL SYSTEMS CORPORATION, Tochigi (JP)

(21) Appl. No.: 18/900,093

(22) Filed: Sep. 27, 2024

(30) Foreign Application Priority Data
Sep. 27, 2023 (JP) 2023-164256
Dec. 18, 2023 (JP) 2023-213405
Dec. 18, 2023 (JP) 2023-213406
Sep. 25, 2024 (JP) 2024-166273

(51) Int. Cl. A61B 8/00; A61B 8/06; A61B 8/14

(52) U.S. Cl. CPC A61B 8/5207; A61B 8/06; A61B 8/14; A61B 8/488

(57) ABSTRACT

An ultrasound diagnostic apparatus according to an embodiment includes a processing circuit. The processing circuit is configured to acquire first signal data of multiple frames through scan on a subject, extract second signal data of multiple frames by extracting a signal by nonlinear processing from the first signal data, identify an inappropriate signal point or an inappropriate frame from the second signal data, and generate third signal data based on an identification result of the inappropriate signal point or the inappropriate frame, and on the second signal data.

### CROSS-REFERENCE TO RELATED APPLICATIONS

This application is based upon and claims the benefit of priority from Japanese Patent Application No. 2023-164256, filed on Sep. 27, 2023; Japanese Patent Application No. 2023-213405, filed on Dec. 18, 2023; Japanese Patent Application No. 2023-213406, filed on Dec. 18, 2023; and Japanese Patent Application No. 2024-166273, filed on Sep. 25, 2024. The entire contents of all of the above-identified applications are incorporated herein by reference.

### FIELD

Embodiments described herein relate generally to an ultrasound diagnostic apparatus, an image processing apparatus, a medical image-processing apparatus, an ultrasound diagnostic method, and a non-transitory computer-readable recording medium.

### BACKGROUND

As a super-resolution technique for blood flow signals, a method such as super resolution using erythrocytes (SURE) as described in Non-Patent Literature 1, and the like have been known. This method uses a fact that an amplitude distribution characteristic of a speckle pattern in an ultrasonic image and a blood flow image follows the Rayleigh distribution. That is, by using the SURE method to accumulate high-strength signals that occur with low probability according to the Rayleigh distribution in each frame, it is possible to obtain blood flow signals with higher resolution compared to conventional methods such as the power Doppler method.

However, during the process of extracting high-strength signals that occur with low probability according to the Rayleigh distribution in each frame, there is a possibility of mistakenly picking up a signal other than blood flow signals, such as a residual tissue signal from clutter or the like, and a motion artifact. In such a case, signals originating from clutter can spread around the blood flow, to reduce the visibility of the blood flow.

### BRIEF DESCRIPTION OF THE DRAWINGS

FIG. 1 is a diagram explaining about an example of a configuration of an ultrasound diagnostic apparatus according to a first embodiment;

FIG. 2 is a flowchart explaining about an example of a flow of processing performed by the ultrasound diagnostic apparatus according to the first embodiment;

FIG. 3A is a diagram explaining about processing performed by the ultrasound diagnostic apparatus according to the first embodiment;

FIG. 3B is a diagram explaining about processing performed by the ultrasound diagnostic apparatus according to the first embodiment;

FIG. 4 is a diagram explaining about processing performed by the ultrasound diagnostic apparatus according to the first embodiment;

FIG. 5A is a diagram illustrating an example of an ultrasonic image by a comparative method;

FIG. 5B is a diagram illustrating an example of an ultrasonic image output by the ultrasound diagnostic apparatus according to the first embodiment;

FIG. 6 is a flowchart explaining an example of a flow of processing performed by an ultrasound diagnostic apparatus according to a second embodiment;

FIG. 7 is a diagram explaining about processing performed by the ultrasound diagnostic apparatus according to the second embodiment;

FIG. 8A is a diagram illustrating an example of an ultrasonic image by the comparative method;

FIG. 8B is a diagram illustrating an example of an ultrasonic image obtained by the ultrasound diagnostic apparatus according to the second embodiment;

FIG. 9 is a diagram illustrating an example of a configuration of an ultrasound diagnostic apparatus according to a third embodiment;

FIG. 10 is a diagram explaining about details of an image generating function 111 according to the third embodiment;

FIG. 11 is a diagram explaining about a background according to the third embodiment;

FIG. 12 is a flowchart illustrating a flow of processing performed by a medical information processing apparatus according to the third embodiment;

FIG. 13A is a diagram explaining about processing performed by the medical information processing apparatus according to the third embodiment;

FIG. 13B is a diagram explaining about processing performed by the medical information processing apparatus according to a comparative example;

FIG. 13C is a diagram explaining about processing performed by the medical information processing apparatus according to the third embodiment;

FIG. 14 is a diagram explaining about an example of processing performed by the medical information processing apparatus according to the third embodiment;

FIG. 15 is a flowchart illustrating a flow of processing performed by the medical information processing apparatus according to a modification of the third embodiment;

FIG. 16A is an example of an ultrasonic image generated by the medical information processing apparatus according to a first comparative example;

FIG. 16B is an example of an ultrasonic image generated by the medical information processing apparatus according to a second comparative example;

FIG. 16C is an example of an ultrasonic image generated by the medical information processing apparatus according to an embodiment;

FIG. 17A is an example of an ultrasonic image generated by the medical information processing apparatus according to a first comparative example;

FIG. 17B is an example of an ultrasonic image generated by the medical information processing apparatus according to a second comparative example;

FIG. 17C is an example of an ultrasonic image generated by the medical information processing apparatus according to the embodiment;

FIG. 18 is a diagram explaining about other embodiments;

FIG. 19 is a diagram explaining about details of an image generating function 111 according to a fourth embodiment;

FIG. 20 is a flowchart illustrating a flow of processing performed by a medical information processing apparatus according to the fourth embodiment;

FIG. 21 is a flowchart explaining a further detailed flow of the processing at step S400 in FIG. 20;

FIG. 22 is a diagram explaining about processing performed by the medical information processing apparatus according to the fourth embodiment;

FIG. 23 is a diagram explaining about processing performed by a medical information processing apparatus according to a comparative example;

FIG. 24 is a diagram explaining about processing performed by the medical information processing apparatus according to the fourth embodiment;

FIG. 25 is a diagram explaining about an example of an image output by the medical information processing apparatus according to the comparative example;

FIG. 26 is a diagram explaining about an example of an image output by the medical information processing apparatus according to the comparative example;

FIG. 27 is a diagram explaining about an example of an image output by the medical information processing apparatus according to the fourth embodiment; and

FIG. 28 is a flowchart illustrating a flow of processing performed by the medical information processing apparatus according to a modification of the fourth embodiment.

### DETAILED DESCRIPTION

An ultrasound diagnostic apparatus according to an embodiment includes a processing circuit. The processing circuit is configured to acquire first signal data having multiple frames by scanning a subject, extract second signal data having multiple frames by extracting a signal by a non-linear processing from the first signal data, identify an inappropriate signal point or an inappropriate frame from the second signal data, and generate third signal data based on an identification result of the inappropriate signal point or the inappropriate frame, and the second signal data.

### First Embodiment

Hereinafter, embodiments of an ultrasound diagnostic apparatus, an image processing apparatus, and an ultrasound diagnostic method will be explained in detail referring to the drawings.

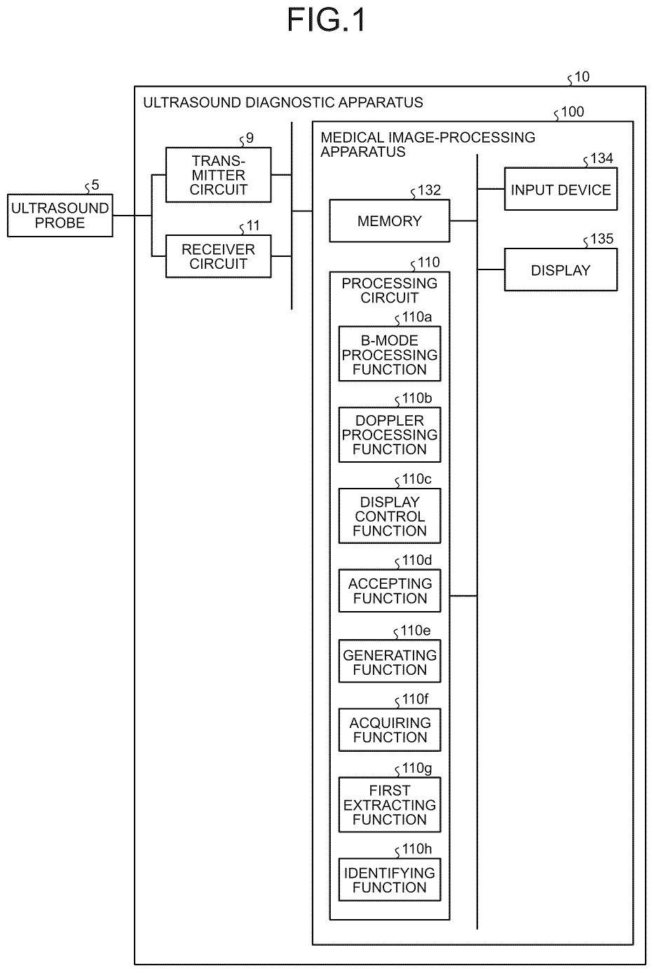

First, a configuration of an ultrasound diagnostic apparatus according to a first embodiment will be explained. FIG. 1 is a block diagram illustrating a configuration example of the ultrasound diagnostic apparatus according to the first embodiment. As illustrated in FIG. 1, the ultrasound diagnostic apparatus according to the first embodiment includes an ultrasound probe 5 and an ultrasound diagnostic apparatus 10, The ultrasound diagnostic apparatus 10 includes a transmitting circuit 9, a receiving circuit 11, and a medical image processing apparatus 100.

The ultrasound probe 5 has multiple piezoelectric transducers, and these piezoelectric transducers generate ultrasonic waves based on a driving signal provided by a transmitting circuit 9 included in the ultrasound diagnostic apparatus 10 described later. Moreover, the piezoelectric transducers included in the ultrasound probe 5 receives a reflected waves from a subject P to convert into an electrical signal (reflection wave signal). Furthermore, the ultrasound probe 5 includes a matching layer that is arranged in a piezoelectric transducer, a backing material to prevent propagation of ultrasonic waves backward from the piezoelectric transducer, and the like. The ultrasound probe 5 is detachably connected to the ultrasound diagnostic apparatus 10.

When ultrasonic waves are transmitted from the ultrasound probe 5 to the subject P, the transmitted ultrasonic waves are sequentially reflected on a discontinuous surface of an acoustic impedance in our body tissue of the subject P, and received by the piezoelectric transducers included in the ultrasound probe 5 as reflected waves, to be converted into a reflection wave signal. An amplitude of the reflection wave signal is dependent on a difference in the acoustic impedance on the discontinuous surface on which the ultrasonic waves are reflected. When transmitted ultrasonic pulses are reflected on a surface of moving blood flow or on a heart wall and the like, the reflection wave signal is subject to frequency shift depending on a velocity component with respect to a direction of transmission of an ultrasonic wave of a moving body due to the Doppler effect.

The embodiment is applicable whether the ultrasound probe 5 is a 1D array probe that scans the subject P in two dimensions or a mechanical 4D probe or a 2D array probe that scans the subject P in three dimensions.

The ultrasound diagnostic apparatus 10 is in an apparatus that generates ultrasonic image data based on the reflection wave signal received from the ultrasound probe 5. The ultrasound diagnostic apparatus 10 illustrated in FIG. 1 is an apparatus that can generate two dimensional ultrasonic image data based on a two-dimensional reflection wave signal, and can generate a three-dimensional ultrasonic image data based on a three-dimensional reflection wave signal. However, the embodiment is applicable even if the ultrasound diagnostic apparatus 10 is an apparatus dedicated for two dimensional data.

The ultrasound diagnostic apparatus 10 includes the transmitting circuit 9, the receiving circuit 11, and the medical image processing apparatus 100 as illustrated in FIG. 1.

The transmitting circuit 9 and the receiving circuit 11 control transmission and reception of ultrasonic waves performed by the ultrasound probe 5 based on an instruction of a processing circuit 110 that has a control function not illustrated. The transmitting circuit 9 provides a driving signal to a pulse generator, a transmission delay unit, a pulser, and the like. The pulse generator repeatedly generates rate pulses at a predetermined pulse repetition frequency (PRF) to form transmission ultrasonic waves. Moreover, the transmission delay unit gives a delay time for each piezoelectric transducer to converge ultrasonic waves generated by the ultrasound probe 5 into a beam shape, and to determine a transmission directivity, to each rate pulse generated by the pulse generator. Furthermore, the pulser applies a driving signal (driving pulse) to the ultrasound probe 5 at a timing based on the rate pulse.

That is, the transmission delay unit varies the delay time to be applied to each rate pulse, and thereby adjusts the transmission direction of an ultrasonic wave to be transmitted from a piezoelectric transducer surface arbitrarily. Moreover, the transmission delay unit controls the position of the focal point (transmission focus) in the depth direction of ultrasonic wave transmission by varying the delay time applied to each rate pulse.

The transmitting circuit 9 has a function of instantly changing a transmission frequency, a transmission drive voltage, and the like to perform a predetermined sequence based on an instruction of the processing circuit 110 described later. Particularly, the change of the transmission drive voltage is implemented by a linear amplifier-type transmission circuit that can instantly switch values, or a mechanism of electrically switching multiple power source units.

Moreover, the receiving circuit 11 includes an amplifier circuit, an analog/digital (A/D) converter, a reception delay circuit, an adder, a quadrature detection circuit, and the like, and generates a reception signal (reflection wave data) by performing various kinds of processing with respect to the reflection wave signal received from the ultrasound probe 5. The amplifier circuit performs gain correction processing by amplifying the reflection wave signal for each channel. The A/D converter converts the reflection wave signal subjected to the gain correction into a digital signal. The reception delay circuit provides a reception delay time necessary for determining a reception directivity for digital data. The adder performs the addition processing of the reflection wave signal that have been given the reception delay time by the reception delay circuit. The addition processing by the adder enhances a reflection component from a direction corresponding to the reception directivity of the reflection wave signal. The quadrature detection circuit converts an output signal from the adder into a baseband in-phase signal (I signal, I: In-phase) and a quadrature-phase signal (Q signal, Q: Quadrature-phase). The quadrature detection circuit then transmits the I signal and @ signal (hereinafter, referred to as IQ signal) to the processing circuit 110 as a reception signal (reflection wave data). The quadrature detection circuit may transmit the output signal of the adder after converting into a radio frequency (RF) signal, to the processing circuit 110. The IQ signal and the RF signal are reception signals having phase information.

When scanning a two-dimensional region in the subject P, the transmitting circuit 9 causes the ultrasound probe 5 to transmit an ultrasound beam to scan the two-dimensional region. The receiving circuit 11 generates a two-dimensional reception signal from the two-dimensional reflection wave signal received from the ultrasound probe 5. Moreover, the transmitting circuit 9 causes the ultrasound probe 5 to transmit an ultrasound beam to scan the three-dimensional region in the subject P. The receiving circuit 11 generates a three-dimensional reception signal from the three-dimensional reflection wave signal received from the ultrasound probe 5. The receiving circuit 11 generates a reception signal based on a reflection wave signal, and transmits the generated reception signal to the processing circuit 110.

The transmitting circuit 9 causes the ultrasound probe 5 to transmit an ultrasound beam from a predetermined transmission position (transmission scan line), The receiving circuit 11 receives a signal based on a reflected wave of the ultrasound beam transmitted by the transmitting circuit 9 at a predetermined reception position (reception scan line) from the ultrasound probe 5. When parallel simultaneous reception is not performed, the transmission scan line and the reception scan line are the same scan line. On the other hand, in the case of performing the parallel simultaneous reception, when the transmitting circuit 9 causes the ultrasound probe 5 to transmit an ultrasonic beam along one transmission scan line, the reception circuit 11 simultaneously receives signals of reflected waves originating from the ultrasound beam that the transmitting circuit 9 has caused the ultrasonic probe 5 to transmit, as multiple reception beams at multiple predetermined reception positions (reception scan lines) through the ultrasonic probe 5.

The medical image processing apparatus 100 is connected to the transmitting circuit 9 and the receiving circuit 11, and performs processing of signals received from the receiving circuit 11 and control of the transmitting circuit 9. The medical image processing apparatus 100 includes the processing circuit 110, a memory 132, an input device 134, and a display 135. The processing circuit 110 includes a B-mode processing function 110a, a Doppler processing function 110b, a display control function 110c, an accepting function 110d, a generating function 110e, an acquiring function 110f, a first extracting function 110g, and an identifying function 110h.

In the embodiment, respective processing functions performed by the B-mode processing function 110a, the Doppler processing function 110b, the display control function 110c, the accepting function 110d, the generating function 110e, the acquiring function 110f, the first extracting function 110g, and the identifying function 110h, and a trained model are stored in the memory 132 in a form of a computer-executable program. The processing circuit 110 is a processor that reads out and executes a program from the memory 132, and thereby implements a function corresponding to each program. In other words, the processing circuit 110 that has read out the respective programs is to have the respective functions indicated in the processing circuit 110 in FIG. 1. Although it has been explained that a function of the processing circuit 110 is implemented by a single processing circuit, the processing circuit 110 may be configured by combining plural independent processors, and may implement the functions with the respective processor executing the respective programs. In other words, each of the functions described above may be configured as a program, and a single processing circuit may execute each program. Furthermore, two or more functions out of the functions of the processing circuit 110 may be implemented by a single processing circuit. As another example, a specific function may be implemented by a dedicated independent program execution circuit.

In FIG. 1, the B-mode processing function 110a, the Doppler processing function 110b, the display control function 110c, the accepting function 110d, the generating function 110e, the acquiring function 110f, the first extracting function 110g, and the identifying function 110h are each examples of a B-mode processing unit, a Doppler processing unit, the display control function 110c, the accepting function 110d, the generating function 110e, the acquiring function 110f, the first extracting function 110g, and the identifying function 110h, respectively.

The term “processor” used in the above explanation signifies a central processing unit (CPU), a graphical processing unit (GPU), or a circuit such as an application specific integrated circuit (ASIC), a programmable logic device (for example, simple programmable logic device (SPLD), complex programmable logic device (CPLD)), and a field programmable gate array (FPGA). The processor implements a function by reading out and executing a program stored in the memory 132.

Instead of storing a program in the memory 132, it may be configured to directly install a program in a circuit of the processor. In this case, the processor reads out and executes the program installed in the circuit, to implement the function.

Moreover, the transmitting circuit 9, the receiving circuit 11, and the like included in the ultrasound diagnostic apparatus 10 can be implemented by hardware, such as an integrated circuit, but it can also be a program modularized in software.

The processing circuit 110 receives data from the receiving circuit 11 by using the B-mode processing function 110a, and performs logarithmic amplification processing, envelope detection processing, logarithmic compression processing, and the like to generate data (B-mode data) in which signal strength is represented by brightness of luminance.

Moreover, the processing circuit 110 performs frequency analysis on the reception signal (reflection wave data) received from the receiving circuit 11 by using the Doppler processing function 110b to extract data (doppler data) that includes moving object information, such as velocity, dispersion, and power, extracted at multiple points, based on the Doppler effect.

The B-mode processing function 110a and the Doppler processing function 110b illustrated in FIG. 1 are capable of processing both two-dimensional reflection wave data and three-dimensional reflection wave data.

The processing circuit 110 controls to display ultrasonic image data for display stored in the memory 132 on the display 135 by using the display control function 110c.

The processing circuit 110 accepts various kinds of operations from a user through the input device 134 by using the accepting function 110d.

The processing circuit 110 generates ultrasonic image data from data generated by the B-mode processing function 110a and the Doppler processing function 110b, by using the generating function 110e. The processing circuit 110 generates two-dimensional B-mode image data in which an intensity of a reflected wave is represented by brightness, from the two-dimensional B-mode data generated by the B-mode processing function 110a, by using the generating function 110e. Moreover, the processing circuit 110 generates two-dimensional Doppler image data that represents the moving object information, from the two-dimensional Doppler data generated by the Doppler processing function 110b, by using the generating function 110e. The two-dimensional Doppler image data is velocity image data, dispersion image data, power image data, and image data obtained by combining these.

Furthermore, the processing circuit 110 converts a scan line signal sequence of ultrasonic scan into a scan line signal sequence in a video format represented by that used for televisions (scan conversion), to generate the ultrasonic image data for display. Moreover, the processing circuit 110 performs image processing to regenerate an average brightness image (smoothing process), image processing that uses a differential filter in an image (edge enhancement processing), and the like as various image processing other than the scan conversion, for example, using multiple image frames after the scan conversion by using the generating function 110e. Furthermore, the processing circuit 110 performs various kinds of rendering processing with respect to volume data to generate two-dimensional image data to display volume data on the display 135.

Moreover, the processing circuit 110 controls overall processing of the ultrasound diagnostic apparatus by a control function not illustrated. Specifically, the processing circuit 110 controls processing of the transmitting circuit 9, the receiving circuit 11, and the processing circuit 110 based on various kinds of setting requests input by an operator through the input device 134, various kinds of control programs and various kinds of data read from the memory 132 by using the control function.

Furthermore, the processing circuit 110 has the respective functions of the acquiring function 110f, the first extracting function 110g, and the identifying function 110h. Details of the respective functions will be described later.

The memory 132 is constituted of a semiconductor memory device, such as a random access memory (RAM) and a flash memory, a hard disk, an optical disk, and the like. The memory 132 is a memory that stores data, such as image data for display generated by the processing circuit 110. Moreover, the memory 132 can store data generated by the B-mode processing function 110a and the Doppler processing function 110b. The B-mode data and the Doppler data stored by the memory 132 can be retrieved by an operator, for example, after diagnosis, and become the ultrasonic image data for display through the processing circuit 110. Furthermore, the memory 132 can store the reception signal (reflection wave data) output by the receiving circuit 11 also.

In addition, the memory 132 stores various kinds of data, such as control programs to perform transmission and reception of ultrasonic waves, image processing, and display processing, diagnosis information (for example, a patient ID, findings of a doctor, and the like), diagnosis protocol, and various kinds of body marks as necessary.

The input device 134 accepts input of various kinds of instructions and information from the operator. The input device 134 is, for example, a pointing device, such as a mouse and a trackball, a selecting device, such as a mode selection switch, or an input device, such as a keyboard.

The display 135 displays a graphical user interface (GUI) to accept an input of an imaging condition, an image generated by the generating function 110e, and the like, under control of the display control function 110c and the like. The display 135 is, for example, a display device, such as a liquid crystal display. The display 135 is one example of a display unit. The display 135 includes a mouse, a keyboard, a button, a panel switch, a touch command screen, a foot switch, a trackball, a joystick, and the like.

Subsequently, a background according to the embodiment will be explained.

As a super-resolution method for a blood flow signal, a method such as the SURE method as described in Non-Patent Literature 1, has been known. This method utilizes the fact that an amplitude distribution characteristic of a speckle pattern in an ultrasonic image and a blood flow image follow a Rayleigh distribution. That is, by using the SURE method to accumulate high-brightness signals that occur with low probability according to a Rayleigh distribution in each frame, across multiple frames, it is possible to obtain a blood flow signal with higher resolution compared to the conventional power Doppler method and the like.

However, there is a possibility of erroneously picking up a signal other than the blood flow signal, such as residual tissue signals of clutter or the like and motion artifacts, in the process of extracting a high brightness signal that occurs with low probability according to the Rayleigh distribution for each frame. In this case, signals originating from clutter and the like can spread around the blood flow, to reduce the visibility of the blood flow.

Based on this background, the ultrasound diagnostic apparatus according to the embodiment includes an acquiring unit, a first extracting unit, an identifying unit, and a generating unit. The acquiring unit acquires first signal data for multiple frames through scanning of a subject. The first extracting unit extracts a signal by the non-linear processing from the first signal data, to extract second signal data of multiple frames. As an example, the first extracting unit extracts the second signal data of multiple frames by extracting a signal with a signal value corresponds to a local maximum value or falls within a predetermined range. The identifying unit identifies an in appropriate signal point or an inappropriate frame from the second signal data. The generating unit generates third signal data based on the identification result of the inappropriate signal point or the inappropriate frame.

Moreover, the image processing apparatus according to the embodiment includes an acquiring unit, a first extracting unit, an identifying unit, and a generating unit. The acquiring unit acquires the first signal data of multiple frames acquired through scanning of a subject. The first extracting unit extracts a signal by the non-linear processing from the first signal data, to extract second signal data of multiple frames. As an example, the first extracting unit extracts the second signal data of multiple frames by extracting a signal with a signal value corresponds to a local maximum value or falls within a predetermined range from the first signal data. The identifying unit identifies an in appropriate signal point or an inappropriate frame from the second signal data. The generating unit generates third signal data by accumulating the second signal data while excluding the inappropriate signal point or the inappropriate frame from an accumulation target.

Furthermore, an ultrasound diagnostic method includes acquiring the first signal data of multiple frames through scanning of a subject, extracting the second signal data of multiple frames by extracting a signal value from the first signal data by the non-linear processing and by extracting, for example, a signal that corresponds to a local maximum value or falls within a predetermined range, and generating the third signal data by identifying an inappropriate signal point or an inappropriate frame from the second signal data and by accumulating the second signal data while excluding the inappropriate signal point or the inappropriate frame from an accumulation target, That is, it is possible to exclude a signal other than that of blood flow by performing an operation to identify an inappropriate signal point or an inappropriate frame from the second signal data after accumulation based on the number of signal point extraction, changes in the number of extracted signals per frame, and the like. Thus, a high-quality blood flow signal from which an inappropriate signal point or frame is removed can be acquired.

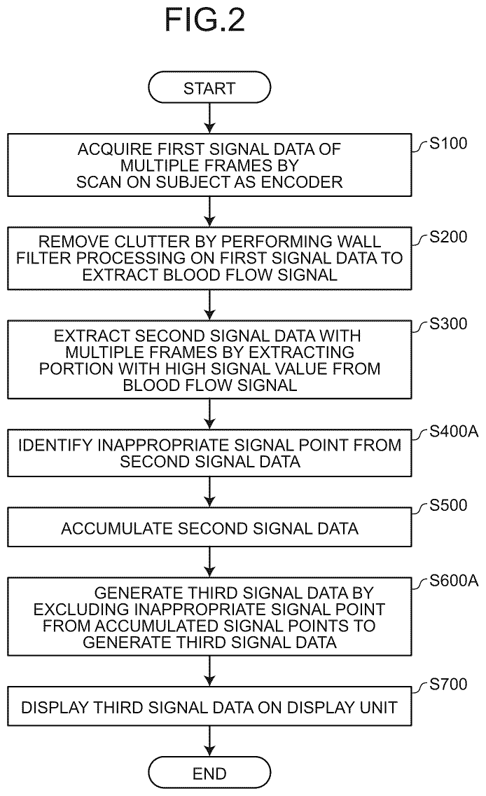

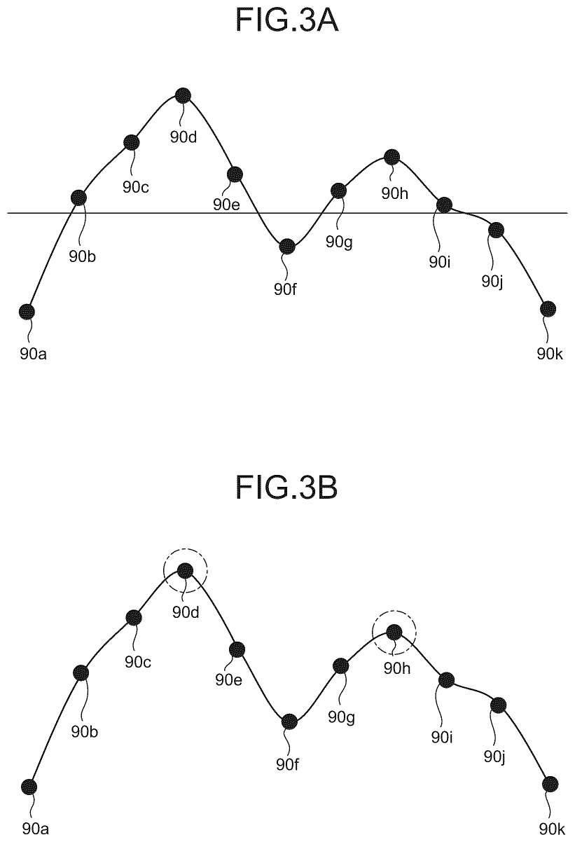

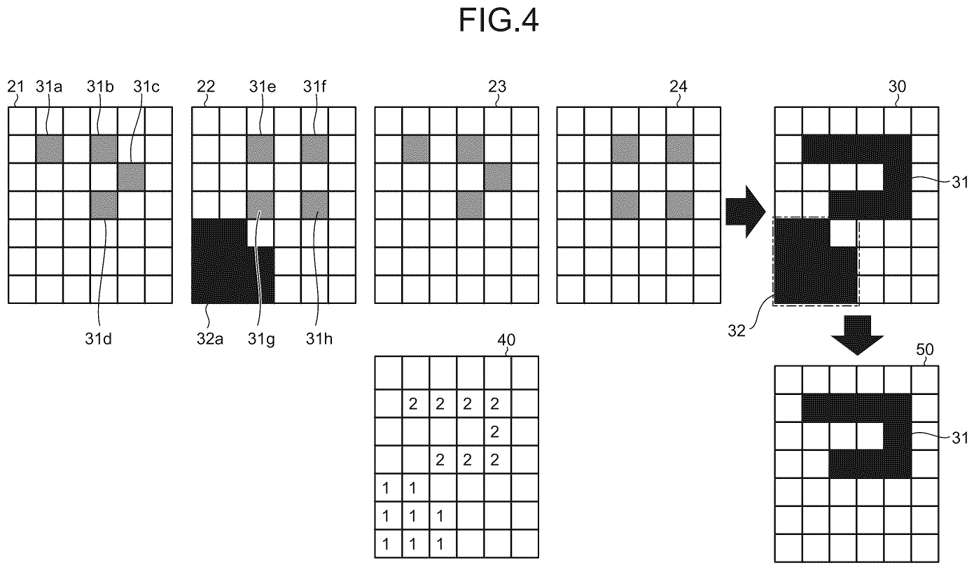

Processing performed by the ultrasound diagnostic apparatus 10 according to the first embodiment will be explained using FIG. 2 in detail referring to FIG. 3A, FIG. 3B, and FIG. 4. FIG. 2 is a flowchart illustrating a flow of the processing performed by the ultrasound diagnostic apparatus 10 according to the first embodiment.

First, at step S100, the transmitting circuit 9 performs ultrasound scan of a subject by transmitting ultrasonic waves to the subject through the ultrasound probe 5. The receiving circuit 11 receives ultrasonic echoes. The processing circuit 110 acquires the first signal data of multiple frames acquired through scanning of the subject from the receiving circuit 11 as echo data by the acquiring function 110f.

Moreover, as another example, the processing circuit 110 may acquire the first signal data by the acquiring function 110f based on signal processing for blood flow imaging. As an example, the processing circuit 110 may acquire signal data obtained by subjecting the signal data of multipleframes acquired through scanning of the subject to the signal processing for blood flow imaging, as the first signal data.

Subsequently, at step S200, the processing circuit 110 removes clutters by performing wall filter processing (MTI filter processing) on the first signal data, and extracts a blood flow signal in which tissue signals are reduced by the first extracting function 110g.

Subsequently, at step S300, the processing circuit 110 extracts the second signal data of multiple frames by extracting a component with a high signal value from the blood flow signal extracted at step S200, by the first extracting function 110g. As an example, the processing circuit 110 extracts the second signal of multiple frames by extracting a signal by the non-linear processing from the first signal data by the first extracting function 110g. As an example, the processing circuit 110 extracts the second signal data of multiple frames by extracting a signal with a signal that corresponds to a local maximum value or falls within a predetermined range, for example, a signal in which a signal value is equal to or higher than a predetermined value, by the first extracting function 110g.

In FIG. 3A and FIG. 3B, the extracting method of a component with a high signal value is specifically illustrated. FIG. 3A illustrates a method of extracting a signal in which a signal value is a predetermined value by performing threshold processing. For simplicity of explanation, the first signal data is depicted as one-dimensional signal data. Points 90a to 90k schematically represent the signal points of each signal data in a single frame, and the vertical direction of a sheet of the figure represents a magnitude of each signal data. The processing circuit 110 extracts the points 90b, 90c, 90d, 90e, 90g, 90h, and 90i having a signal value equal to or higher than a threshold indicated by a line in FIG. 3A by the first extracting function 110g as the second signal data.

On the other hand, FIG. 3B illustrates a case in which the processing circuit 110 extracts the second signal data of multiple frames by performing local-peak detection processing by the first extracting function 110g to extract a signal in which a signal value is a local maximum value from the first signal data. Similarly to FIG. 3A, points 90a to 90k schematically represent each signal data in a single frame, and the vertical direction of a sheet of the figure represents a magnitude of each signal data. In FIG. 3B, the processing circuit 110 extracts the point 90d and the point 90h that corresponds to the local maximum value as the second signal data by the first extracting function 110g.

Although the first signal data is illustrated as one-dimensional signal data in FIG. 3A and FIG. 3B for simplicity of explanation, data of each frame of the first signal data of multiple frames is normally two-dimensional signal data. Even when the first signal data is signal data of two or more dimensions, by performing processing similar to the processing described above, the processing circuit 110 can extract the second signal data of multiple frames by the first extracting function 110g.

FIG. 4 illustrates multiple pieces of the second signal data thus extracted. Frames 21 to 24 represent respective frames of the first signal data of multiple frames, or respective frames of blood-flow signal data after the clutter removal processing is performed on the first signal data of multiple frames at step S200. For example, data of the frame 21 represents the first signal data in a first frame, or blood-flow signal data obtained by performing the clutter removal processing on the first signal data in the first frame. Moreover, data in the frame 22 represents the first signal data in a second frame, or the blood-flow signal data after the clutter removal processing is performed on the second signal data in the second frame.

Moreover, in frames 21 to 24, pixels in gray or black represent the signal data extracted as the second signal data. For example, in FIG. 4, points 31a to 31d are to be signal data extracted as the second signal data in the frame 21, which is the first frame. Furthermore, points 31e, 31f, 31g, 31h, and a region 32a are to be the signal data extracted as the second signal data in the frame 22, which is the second frame. Moreover, among the frames 21 to 24, gray pixels represent signal points that are not extracted as inappropriate signal points at step S400A described later, and black pixels represent signal points that are extracted as inappropriate signal points at step S400A.

Subsequently, at step S400A, the processing circuit 110 identifies the inappropriate signal point from the second signal data extracted at step S300 by the identifying function 110h. The inappropriate signal point is a signal point that is presumed not to be a blood flow signal of the observation target, such as a clutter. Because the clutter and the like are considered unlikely to be extracted as a high signal point across multiple frames at the same position, the processing circuit 110 identifies an inappropriate signal point based on the number of signal extraction across multiple frames by the identifying function 110h. As an example, the processing circuit 110 identifies a signal point at a position at which the number of signal extraction is lower than a criterion as the inappropriate signal point by the identifying function 110h.

For example, in FIG. 4, accumulation signal data 30 represents the second signal data obtained by performing accumulation processing at step S500 described later. Moreover, at step S400A, the processing circuit 110 generates a table 40 to count the number of times each point has been extracted as a high signal point at each position. For example, because points 31a, 31b, 31c, and the like are extracted as a high signal point in more than two frames, the number of signal extraction is to be “2”. On the other hand, because a point belonging to the region 32a is extracted as a high signal point only in the frame 22, which is the second frame, that is, only in a single frame, the number of signal extraction is to be “1”.

The processing circuit 110 sets, for example, the number of signal extraction to be a criterion to “2”. In this case, the processing circuit 110 determines signal points in a region 31 that are signal points, such as the points 31a, 31b, and 31c, the number of signal extraction of which is equal to or larger than 2, as appropriate signal points by the identifying function 110h, On the other hand, the processing circuit 110 identifies the signal point in a region 32, the number of signal extraction of which is 1, as an inappropriate signal point by the identifying function 110h.

In the example described above, a case in which the processing circuit 110 performs the determination processing for inappropriate signal points based on the number of signal extraction using the specific function 110h has been explained. However, the embodiment is not limited thereto. As an example, the processing circuit 110 may use the identifying function 110 to identify an inappropriate signal point at step S300A by using a neural network.

Subsequently, at step S500, the processing circuit 110 generates the accumulation signal data in which data of respective frames are integrated by accumulating the second signal data in multiple frames extracted at step S300, by the generating function 110e. As an example, in FIG. 4, the processing circuit 110 generates the accumulation signal data 30 by accumulating the second signal data that are high signal points in gray or black in the frames 21 to 24 by the generating function 110e.

Subsequently, at step S600A, the processing circuit 110 generates the third signal data from which an inappropriate signal is removed based on the identification result of the inappropriate signal point identified at step S400A and the second signal data extracted at step S300, by the generating function 110e. As an example, the processing circuit 110 generates the third signal data by removing an inappropriate signal from the accumulation signal data 30 in which the second signal data is accumulated, by the generating function 110e. In other words, the processing circuit 110 generates the third signal data by removing the inappropriate signal point from the signal points after accumulation, by the generating function 110e. As an example, in FIG. 4, the processing circuit 110 removes a signal point in the region 32 that is a signal point at a position at which the number of signal extraction is smaller than the criterion as the inappropriate signal point from the accumulation signal data 30 in which the second signal data is accumulated, and generates the third signal data in which only appropriate signal points in the region 31 are accumulated. The embodiment is not limited thereto, and the processing circuit 110 may generate the third signal data by accumulating the second signal data while removing an inappropriate signal point from the accumulation target by the generating function 110e.

In the embodiment described above, a case in which the processing circuit 110 removes an inappropriate signal point by the generating function 110e has been explained, but the embodiment is not limited thereto. The processing circuit 110 may generate the third signal data while reducing a signal intensity of an inappropriate signal point by the generating function 110e.

At step S400A to step S600A, as for the criterion to identify an inappropriate signal point and a method of generating the third signal data, it may be configured to perform automatic setting by the ultrasound diagnostic apparatus 10, and to remove an in appropriate signal automatically by the processing circuit 110, not based on an input by a user.

Subsequently, at step S700, the processing circuit 110 causes the display 135 serving as a display unit to display the third signal data by the display control function 110c. Thus, a blood-flow signal image with a high image quality from which a signal other than that of blood flow is removed can be acquired.

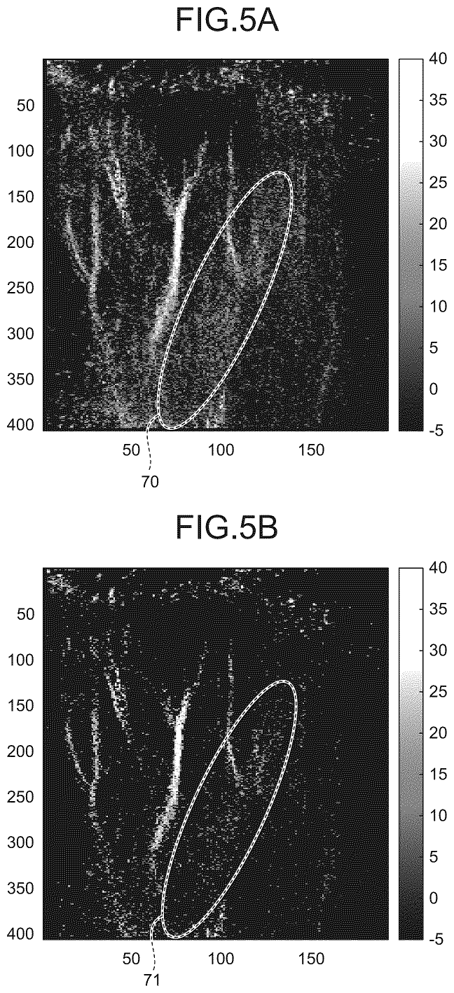

FIG. 5A is a diagram illustrating an example of an ultrasonic image in a comparative method. On the other hand, FIG. 5B is a diagram illustrating an example of an ultrasonic image generated by the ultrasound diagnostic apparatus 10 according to the first embodiment. As illustrated in FIG. 5A, in an ultrasonic image by the comparative method, in a region 70, a signal other than a blood flow signal is included. On the other hand, as illustrated in FIG. 5B, in an ultrasonic image generated by the ultrasound diagnostic apparatus according to the first embodiment, a signal other than a blood flow signal has been removed in a region 71, which is the same region as the region 70.

As described above, in the first embodiment, the processing circuit 110 generates the third signal data by identifying an inappropriate signal point based on the number of signal extraction by the identifying function 110h. Thus, a signal point to be a noise can be removed, and the image quality can be improved.

### Modification of First Embodiment

The embodiment is not limited to the example described above.

In the example described above, a case in which the criterion for identifying an inappropriate signal point by the processing circuit 110 using the first extracting function 110g at step S400A to step S600A is automatically determined by the ultrasound diagnostic apparatus 10 has been explained, but the embodiment is not limited thereto. As an example, the processing circuit 110 may be configured to accept input of the criterion to identify an inappropriate signal point or an inappropriate frame from a user by the accepting function 110d, and to cause the display 135 serving as the display unit to display a value of an index value for each position or each frame for the user to set the criterion by the display control function 110c.

As an example, at step S400A, the processing circuit 110 measures the number of times each point has been extracted as a high signal point at each point by the identifying function 110h by the display control function 110c, and generates a table 40 illustrated in FIG. 4 indicating the number of times of signal point extraction at each point. Subsequently, the processing circuit 110 causes the display 135 serving as the display unit to display information corresponding to the table 40 in which the number of times of signal point extraction at each point is indicated, by the display control function 110c. Furthermore, the processing circuit 110 accepts input of a criterion to identify an inappropriate signal point from the user, by the accepting function 110d. The processing circuit 110 identifies an inappropriate signal point from the second signal data at step S400A based on the criterion input by the user, by the identifying function 110h.

Furthermore, in the embodiment, the case in which the processing circuit 110 identifies an inappropriate signal based on the number of signal extraction at each point by the identifying function 110h at step S400A has been explained, but the embodiment is not limited thereto. As an example, the processing circuit 110 may be configured to identify an inappropriate signal point based on a signal intensity at each position by the identifying function 110h. For example, the processing circuit 110 identifies a signal point, the signal intensity at each position of which is lower than a criterion as an inappropriate signal point, by the identifying function 110h.

### Second Embodiment

In the first embodiment, the case in which the processing circuit 110 identifies an inappropriate signal point by the identifying function 100h at step S400A, and generates the third signal data by removing the identified inappropriate signal point from signal points after accumulation by the generating function 110e at step S600A has been explained. In the second embodiment, a case in which the third signal data is generated not by identifying an inappropriate signal point, but by identifying an inappropriate frame will be explained.

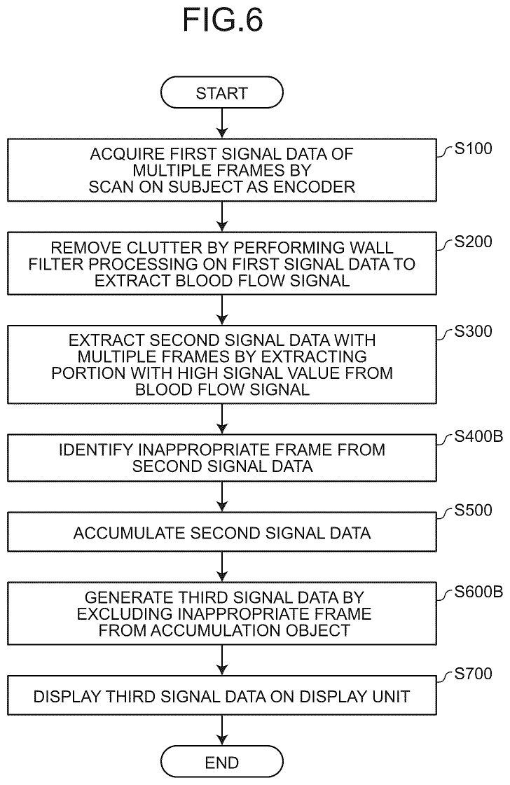

FIG. 6 is a flowchart illustrating a flow of processing performed by the ultrasound diagnostic apparatus according to the second embodiment. Processing other than that at step S400B and step S600B is the same as that of the first embodiment and, therefore, duplicated explanation will be omitted.

At step S400B, the processing circuit 110 identifies an inappropriate frame from the second signal data extracted at step S300, by the identifying function 110h. The inappropriate frame is a frame that is estimated to include a signal point presumed not to be a blood flow signal of the observation target, such as a clutter. It is expected that the number of extracted signals identified as a high signal point changes rapidly in the frame direction near a frame in which the clutter is occurring. Therefore, the processing circuit 110 identifies an inappropriate frame based on the number of extracted signals in the frame direction by the identifying function 110h. As an example, the processing circuit 110 identifies a frame in which the number of extracted signals in the frame direction is larger than a criterion as an inappropriate frame by the identifying function 110h.

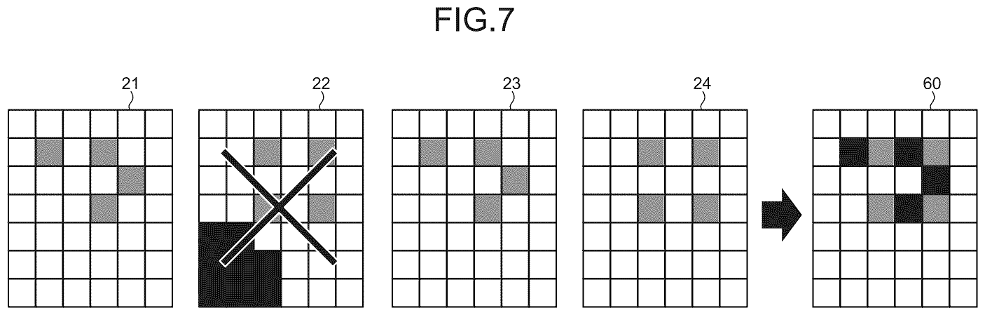

For example, in FIG. 7, the frames 21 to 24 represent the respective frames of the first signal data of multiple frames. In frames 21 to 24, the signal points in gray or black represent signal points with high signal values extracted as an area with a high signal value by the processing circuit 110 through the first extracting function 110g at step S300. At step S400B, the processing circuit 110 identifies a frame in which the change in the number of extracted signals in the frame direction is larger than the criterion as an inappropriate frame by the identifying function 110h. In the example in FIG. 7, the number of signal points extracted as the signal point with high signal value in the frame 22 is “12”. On the other hand, in the frames 21 and 23, the number of signal points extracted as signal points with a high signal value is “4”. Therefore, because the change in the number of extracted signals in the frame direction exceeds the criterion near the frame 22, the processing circuit 110 identifies the frame 22 as an inappropriate frame, by the identifying function 110h. In the example described above, a case in which the processing circuit 110 performs the determination processing for an inappropriate frame based on the change in the number of extracted signals in the frame direction by the identifying function 110h has been explained, but the embodiment is not limited thereto. As an example, the processing circuit 110 may identify an inappropriate signal point at step S400B based on a comprehensive determination criterion using a neural network, by the identifying function 110h.

At step S600B, the processing circuit 110 generates the third signal data from which an inappropriate frame has been removed based on the identification result of an inappropriate frame identified at step S400B and the second signal data identified at step S300. As an example, the processing circuit 110 generates the third signal data by accumulating the second data while removing an inappropriate frame from the accumulation target by the generating function 110e. As an example, in FIG. 7, the processing circuit 110 generates third signal data 60 by accumulating the second signal data while removing the frame 22, which is an inappropriate frame, from the accumulation target by the generating function 110e.

In the embodiment described above, a case in which the processing circuit 110 extracts an inappropriate frame based on the change in the number of extracted signals in the frame direction at step S400B by the identifying function 110h has been explained, but the embodiment is not limited thereto. The processing circuit 110 may identify an inappropriate frame based on the number of extracted signals in the frame direction itself at step S400B by the identifying function 110h. As an example, the processing circuit 110 may identify a frame in which the number of extracted signals in the frame direction exceeds a threshold by the identifying function 110h.

Moreover, at step S400B to step S600B, as for the criterion to identify an inappropriate signal point and a method of generating the third signal data, it may be configured to perform automatic setting by the ultrasound diagnostic apparatus 10, and to remove an in appropriate signal automatically by the processing circuit 110, not based on an input by a user.

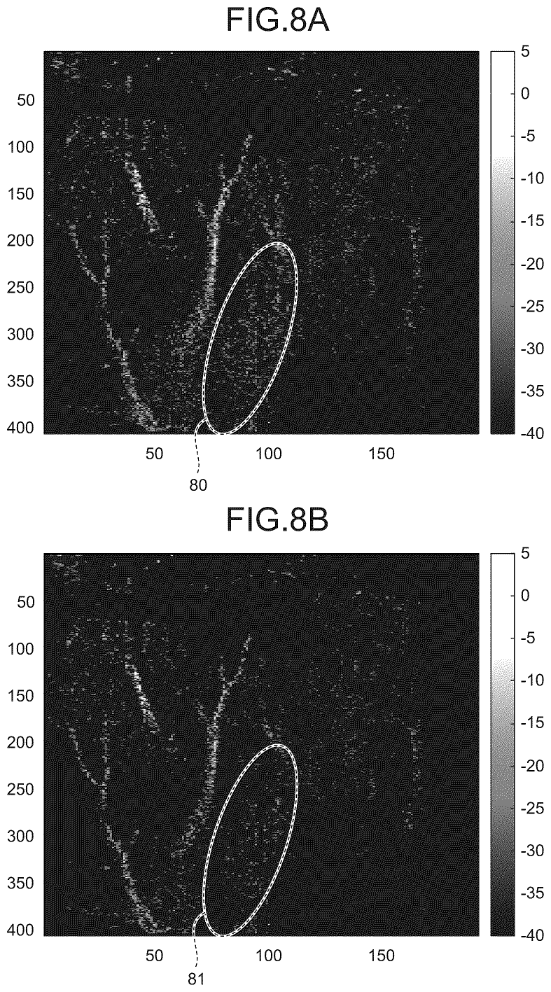

FIG. 8A is a diagram illustrating an example of an ultrasonic image by a comparative method. On the other hand, FIG. 8B is a diagram illustrating an example of an ultrasonic image generated by the ultrasound diagnostic apparatus 10 according to the second embodiment. As illustrated in FIG. 8A, in the ultrasonic image by the comparative example, a signal other than a blood flow signal is included in a region 80. On the other hand, as illustrated in FIG. 8B, in an ultrasonic image generated by the ultrasound diagnostic apparatus 10 according to the second embodiment, a signal other than a blood flow signal has been removed in a region 81, which is the same region as the region 80.

As described above, in the second embodiment, the processing circuit 110 generates the third signal data by identifying an inappropriate frame based on the change in the number of extracted signals in the frame direction by the identifying function 110h. Thus, a frame to be a noise can be removed, and the image quality can be improved.

### Third Embodiment

Hereinafter, for a third embodiment, an embodiment of a medical-information processing apparatus, an ultrasound diagnostic apparatus, and a program will be explained in detail referring to the drawings.

First, definitions of several terms used in this specification will be explained briefly for the third embodiment.

The ultrasound diagnostic apparatus according to the third embodiment causes an ultrasound probe to perform an ultrasound scan, and collects multiple pieces of frame data (multiple pieces of frame data within a predetermined time) in a sequence in time that are acquired by execution of the ultrasound scan. The frame data is collected at a predetermined frame rate through the execution of an ultrasound scan, and refers to either one of reception image, measurement image, and blood flow image. The reception image represents a reception signal of an ultrasonic wave received by the ultrasound probe in a two-dimensional image format. The measurement image represents measurement data acquired by performing coherent addition and quadrature detection on the reception signal in a two-dimensional image format. The blood flow image represents blood flow data extracted or enhanced from the measurement data to emphasize information derived from blood flow in a two-dimensional image format.

The measurement data includes information derived from a tissue (tissue image component) and information derived from blood flow (blood flow image component). The information derived from blood flow may include not only information derived from blood, but also information derived from a contrast agent in blood. Moreover, the blood flow data is data including a pixel value extracted or enhanced from the information derived from blood flow, and includes a flow velocity, a dispersion value, and a power value.

Extraction of information derived from blood flow is, for example, an operation of selectively extracting image characteristics of a blood-flow image component from the measurement data. Enhancement of the information derived from blood flow is, for example, an operation of relatively emphasizing the image characteristic of the blood-flow image component to image characteristics of the tissue image component. The blood flow data may be acquired by processing of extracting or enhancing the information derived from blood flow, or by processing of removing or reducing the information derived from the tissue.

Furthermore, the ultrasound diagnostic apparatus performs processing of successively extracting image components representing a target object from first ultrasonic-image data described later. The target object includes, for example, either one of blood, a body tissue, and a contrast agent.

The ultrasound diagnostic apparatus output second ultrasonic-image data based on image components representing the target object that are successively extracted from the first ultrasonic-image data. The second ultrasonic-image data may be either one of composite image data that is obtained by performing composition processing on the successively extracted image components representing the target object and the first ultrasonic image data, corrected image data that is obtained by performing correction processing on the first ultrasonic-image data based on the successively extracted image components representing the target object, and image data that includes the successively extracted image components representing the target object. According to the ultrasound diagnostic apparatus according to the present embodiment, even if the number of images used for extraction of a target object is small, image components (pixel values) representing the target object can be successively extracted, and a highly responsive and high-resolution ultrasonic image can be obtained. Moreover, according to the ultrasound diagnostic apparatus according to the present embodiment, compared to a conventional apparatus, the computational load for signal extraction can be reduced to 1/frame count, thereby lowering the overall computational load of signal extraction.

Although an example in which the present embodiment is applied to an ultrasound diagnostic apparatus has so far been explained, the present embodiment may also be applied to another modality (medical information processing apparatus) other than the ultrasound diagnostic apparatus. For example, it can be applied to a medical information processing apparatus such as a workstation and a server that acquires ultrasonic image data acquired based on a result of ultrasound scan of a subject.

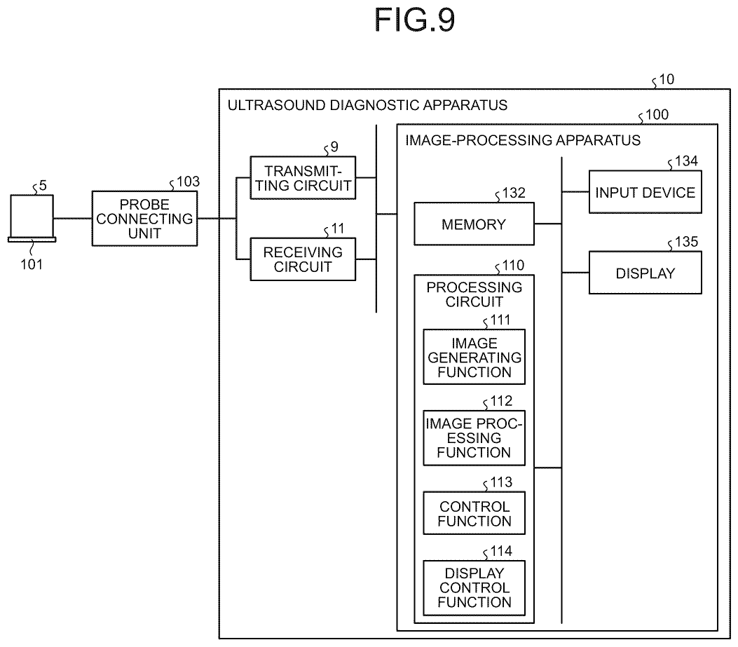

FIG. 9 is a block diagram illustrating a configuration example of an ultrasound diagnostic apparatus according to a third embodiment. The ultrasound diagnostic apparatus 10 is an apparatus that generates ultrasonic image data based on a reflection wave signal received from the ultrasound probe 5. The ultrasound diagnostic apparatus 10 illustrated in FIG. 9 is an apparatus that is capable of generating two-dimensional ultrasonic image data based on a two-dimensional reflection wave signal, and of generating three-dimensional ultrasonic image data based on a three-dimensional reflection wave signal. However, the embodiment is applicable even when the ultrasound diagnostic apparatus 10 is an apparatus dedicated for two-dimensional data. The ultrasound diagnostic apparatus 10 includes the transmitting circuit 9, the receiving circuit 11, and the image processing apparatus 100 serving as the medical information processing apparatus according to the embodiment.

The ultrasound probe 5 is, for example, an electronic scanning probe, and has multiple transducers 101 arranged in one-dimensional or two-dimensional configuration at its distal end. The transducer 101 is a piezoelectric device (electromechanical conversion element) that performs mutual conversion between an electrical signal (voltage pulse signal) and an ultrasonic wave (acoustic wave), The ultrasound probe 5 transmits an ultrasonic wave from the multiple transducers 101 to a subject, and receives a reflected ultrasonic wave from the subject by the transducers 101. A reflected acoustic wave reflects a difference in acoustic impedance in the subject. When transmitted ultrasonic pulses are reflected on a surface of moving blood flow or on a heart wall and the like, the reflection ultrasound signal is subject to frequency shift depending on a velocity image component with respect to a direction of transmission of an ultrasonic wave of a moving body due to the Doppler effect.

A probe connecting unit 103 connects the ultrasound probe 5, and performs transmission and reception of ultrasonic waves with the ultrasound probe 5. A method of connecting the ultrasound probe 5 by the probe connecting unit 103 may be any of wired and wireless connections. In the case of wired connection, the probe connecting unit 103 has a connector portion (receptacle) to connect a connector (plug) of the ultrasound probe 5. In the case of wireless connection, a communication unit that performs wireless communication with the ultrasound probe 5 is included.

The transmitting circuit 9 is a transmitting unit that outputs a pulse signal (driving signal) to the multiple transducers 101. By applying pulse signals with a time delay to the transducers 101, ultrasonic waves with different delay times are transmitted from the multiple transducers 101, to form a transmission ultrasound beam. By selectively changing the transducer 101 to which the pulse signal is applied (that is, the transducer 101 to be driven), or by varying the delay time of the pulse signal (application timing), the direction and focus of the transmission ultrasound beam can be controlled. By sequentially changing the direction and the focus of this transmission ultrasound beam, an observation region inside the subject is scanned. Moreover, by changing the delay time of the pulse signal, a transmission ultrasonic beam that is a plane wave (with a far-field focus) or a divergent wave (with a focal point opposite to the ultrasonic transmission direction for the multiple transducers 101) may be formed. Alternatively, an ultrasound beam may be formed using one transducer or some of the multiple transducers 101. The transmitting circuit 9 transmits a pulse signal of a predetermined driving waveform to the transducer 101, to generate a transmission ultrasonic wave having a predetermined waveform in the transducer 101. The receiving circuit 11 is a receiving unit that receives an electrical signal output from the transducer 101 that has received a reflected ultrasonic wave as a reception signal. The reception signal is input to an image generating unit of the processing circuit 110.

Operation of the transmitting circuit 9 and the receiving circuit 11, that is, transmission and reception of an ultrasonic wave are controlled by control function 113 of the processing circuit 110. The processing circuit 110 changes a position at which a voltage signal or a transmission ultrasonic wave is formed, for example, according to each generation of a B-mode image and a blood flow image described later by the control function 113. The transmitting circuit 9 and the receiving circuit 11 are one example of an executing unit.

To generate a B-mode image, a reception signal of a reflected ultrasonic wave acquired by scanning an observation region is acquired to be used. To generate a blood flow image, a reception signal of a reflected ultrasonic wave of multiple frames is acquired by performing transmission and reception of ultrasonic waves on each of a single or multiple scan lines in the observation region multiple times, to be used for image generation, that is, extraction of blood flow information. For scanning to generate a blood flow image, it may be a method in which transmission and reception are performed multiple times on one scan line and then transmission and reception are performed on a next scan line, or may be a method in which transmission and reception is performed one time on each scan line and this is repeated multiple times. Moreover, in generation of a B-mode image and a blood flow image, ultrasonic waves may be transmitted in a wide range of the observation region, so as to reduce the number of scan lines, that is, by transmitting a plane wave or a divergent wave. Furthermore, the transmission and reception may be performed multiple times to a wide range of the observation region by varying a transmission angle of the plane wave or divergent wave, or a range of the observation region to which the waves are transmitted, and the reception signals may be summed for use. In the present specification, both an analog signal output from the transducer 101 and digital data obtained by sampling it (digital conversion) are referred to as reception signal, not particularly distinguishing from each other. However, depending on the context, the reception signals may also be referred to as reception data or measurement data for the purpose of explicitly indicating it is digital data.

The image processing apparatus 100 is connected to the transmitting circuit 9 and the receiving circuit 11, and performs a process of a signal received from the receiving circuit 11 and control of the transmitting circuit 9. The image processing apparatus 100 includes the processing circuit 110, the memory 132, the input device 134, and the display 135. The processing circuit 110 includes the image generating function 111, image processing function 112, the control function 113, and display control function 114.

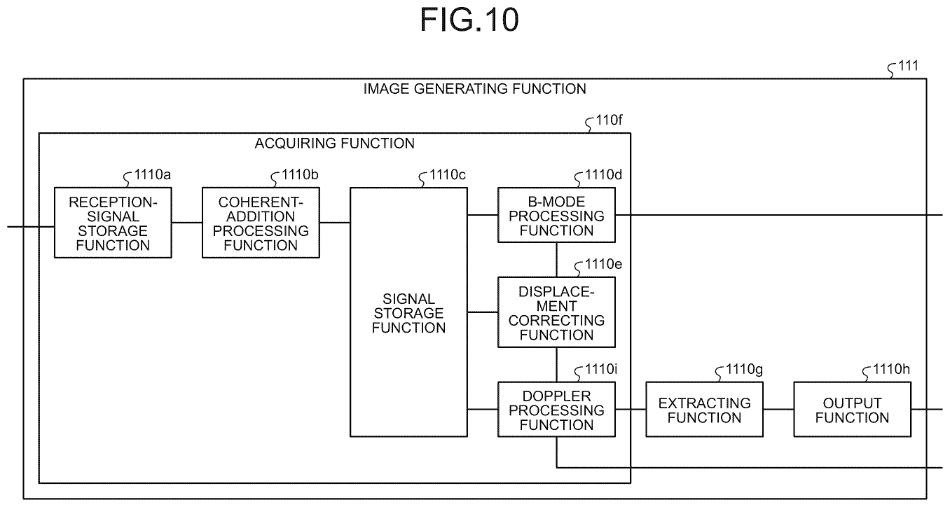

Moreover, as illustrated in FIG. 10, more specifically, the image generating function 111 includes the acquiring function 110f, the extracting function 1110g, and the output function 1110h. More specifically, the acquiring function 110f is constituted of a reception-signal storage function 1110a, a coherent-addition processing function 1110b, a signal storage function 1110c, a B-mode processing function 1110d, a displacement correcting function 1110e, a Doppler processing function 1110i, and the like.

In the embodiment, respective processing functions performed by the reception-signal storage function 1110a, the coherent-addition processing function 1110b, the signal storage function 1110c, the B-mode processing function 1110d, the displacement correcting function 1110e, the acquiring function 110f, the extracting function 1110g, the output function 1110h, the Doppler processing function 11101, the image generating function 111, the image processing function 112, the control function 113, the display control function 114, and the like, and a trained model are stored in the memory 132 in a form of a computer-executable program. The processing circuit 110 is a processor that implements a function corresponding to each program by reading out and executing the program stored in the memory 132. In other words, the processing circuit 110 that has read the respective programs are to have the respective functions indicated in the processing circuit 110 in FIG. 9 or the image generating function 111 in FIG. 10. The functions of the processing circuit 110 is explained as being implemented by a signal processing circuit in FIG. 1, but the processing circuit 110 may be configured by combining multiple independent processors, to implement the functions by the respective processors executing the programs. In other words, the respective functions described above are configured as programs, and one processing circuit may be configured to execute each program. Moreover, two or more functions out of the functions included in the processing circuit 110 may be implemented by a single processing circuit. As another example, specific functions may be implemented in dedicated independent program execution circuit.

In FIG. 9 and FIG. 10, the image generating function 111, the image processing function 112, the control function 113, the display control function 114, the acquiring function 110f, the extracting function 1110g, and the output function 1110h are one example of an image generating unit, a processing unit, a control unit, a display control unit, an acquiring unit, an extracting unit, and an output unit, respectively.

The term “processor” used in the above explanation signifies a circuit such as a CPU, similarly to the first embodiment. The processor implements a function by reading out and executing a program stored in the memory 132.

Moreover, instead of storing a program in the memory 132, it may be configured to directly install a program in a circuit of the processor. In this case, the processor reads out and executes the program installed in the circuit, to implement the function.

Moreover, the transmitting circuit 9, the receiving circuit 11, and the like included in the ultrasound diagnostic apparatus 10 can be implemented by hardware, such as an integrated circuit, but it can also be a program modularized in software.

The processing circuit 110 generates image data based on the reception signal acquired from the ultrasound probe 5 by the image generating function 111. Details of the image generating function 111 will be explained later. The processing circuit 110 subjects image data generated by reception-signal processing unit 106 to image processing, such as brightness adjustment, interpolation, and filtering, by the image processing function 112. The processing circuit 110 is a control unit that comprehensively controls the transmitting circuit 9, the receiving circuit 11, the image processing function 112, the display 135, and the like by the control function 113. The processing circuit 110 controls display of image data and various kinds of information by the display control function 114.

The memory 132 is constituted of a semiconductor memory device, such as a RAM and a flash memory, a hard disk, an optical disk, and the like. The memory 132 is a memory that stores data, such as image data for display generated by the processing circuit 110. Moreover, the memory 132 can store data generated by the B-mode processing function 1110d and the Doppler processing function 11101. The B-mode data and the Doppler data stored by the memory 132 can be retrieved by an operator, for example, after diagnosis, and become the ultrasonic image data for display through the processing circuit 110. Furthermore, the memory 132 can store the reception signal (reflection wave data) output by the receiving circuit 11 also.

In addition, the memory 132 stores various kinds of data, such as control programs to perform transmission and reception of ultrasonic waves, image processing, and display processing, diagnosis information (for example, a patient ID, findings of a doctor, and the like), diagnosis protocol, and various kinds of body marks as necessary.

The input device 134 accepts input of various kinds of instructions and information from the operator. The input device 134 is, for example, a pointing device, such as a mouse and a trackball, a selecting device, such as a mode selection switch, or an input device, such as a keyboard.

The display 135 displays a GUI to accept an input of an imaging condition, an image generated by the output function 1110h, and the like, under control of the display control function 114 and the like. The display 135 is, for example, a display device, such as a liquid crystal display. The display 135 is one example of a display unit. The display 135 includes a mouse, a keyboard, a button, a panel switch, a touch command screen, a foot switch, a trackball, a joystick, and the like,

### Configuration of Reception-Signal Processing Block

FIG. 10 is a block diagram illustrating an example of the image generating function 111. The image generating function 111 is constituted of the acquiring function 110f, the extracting function 1110g, and the output function 1110h. Moreover, the acquiring function 1110f is constituted of the reception-signal storage function 1110a, the coherent-addition processing function 1110b, the signal storage function 1110c, the B-mode processing function 1110d, the displacement correcting function 1110e, and the Doppler processing function 1110i. The reception-signal storage function 1110a stores the reception signal received by the receiving circuit 11. It may be configured not to store the reception signal received by the receiving circuit 11 by the reception-signal storage function 1110a depending on a device configuration and a type of reception signal, but to store by the signal storage function 1110c after the coherent addition processing function 1110b described later. Moreover, the reception-signal storage function 1110a may be constituted of a common block as the signal storage function 1110c described later, and may store the reception signal from the receiving circuit 11 and the reception signal after the coherent-addition processing function 1110b.

The coherent-addition processing function 1110b performs the coherent addition and quadrature detection on the reception signal acquired by the receiving circuit 11, and stores the reception signal subjected to the processing by the signal storage function 1110c. The coherent addition processing is processing to form a reception ultrasound beam by varying the delay time and weight for each transducer 101, and by combining the reception signals from multiple transducers 101, and is called delay and sum (DAS) beam forming also. The quadrature detection processing is processing to convert the reception signal into a baseband in-phase signal (I signal) and a quadrature-phase signal (Q signal). The coherent addition processing and the quadrature detection processing are performed based on a device arrangement input by the control function 113 and various conditions (aperture control, signal filtering). The reception signal after subjected to the coherent addition processing and the quadrature detection processing is stored by the signal storage function 1110c. Although an example of typical DAS beamforming is described, any type of processing for forming a reception ultrasound beam, such as adaptive beamforming, model-based processing, or processing using machine learning, may be used. The reception signal (digital data) acquired by the receiving circuit 11 is also called RAW data. The reception signal (digital data) acquired by the coherent-addition processing function 1110b is also called RF data. The RAW data and the RF data are one example of measurement data acquired based on a reception signal of an ultrasonic wave reflected in a living body.

The B-mode processing function 1110d generates image data in which a signal intensity is expressed by brightness intensity at each point in an observation region by performing envelope detection processing, logarithmic compression processing, and the like on the reception signal for B-mode image generation stored by the signal storage function 1110c. Moreover, the B-mode processing function 1110d may perform the B-mode processing on the reception signal in which the displacement is corrected by the displacement correcting function 1110e.

The displacement correcting function 1110e calculates an amount of displacement of a tissue due to body movement among frames from the reception signals acquired from multiple frames. Generally, it is calculated using block-matching operations referred to as speckle tracking method. By setting a region of interest within a frame, and by tracking the region of interest across frames using correlation, the displacement of the region of interest is calculated. By setting multiple regions of interest within a frame, the displacement for the entire frame is calculated. Although an example using correlation between frames is described herein, any method that can determine the displacement of the region of interest can be used. In the ultrasonic wave signal, because tracking of speckle, which is the scattered image of ultrasonic wave reflected from a scatterer in a body tissue, is performed, it is called speckle tracking. Correlation calculations of the region of interest between frames can be performed on the reception signals after any one of the coherent addition processing, the quadrature detection processing, and the envelope detection processing. Moreover, the correlation calculation may be performed on a time waveform of the reception signal or on a frequency-domain data obtained by performing a discrete Fourier transform on the reception signal. The displacement correcting function 1110e corrects the displacement by moving the reception signal with respect to a reference frame using a calculated displacement. The displacement to be corrected may be set as a uniform value for the entire frame, such as an average displacement of each region of interest, or it may vary within the frame for each region of interest. Furthermore, the displacement of the entire frame calculated for each region of interest may be interpolated in a frame or in a temporal direction of data sequence using linear interpolation, spline interpolation, or the like. Moreover, when correcting the displacement, to correct more precisely, the reception signal may be moved similarly after interpolation.

The Doppler processing function 1110i extracts blood flow data from the reception signal for blood flow image generation stored by the signal storage function 1110c. Moreover, the Doppler processing may be performed, by the Doppler processing function 1110i, with respect to the reception signal in which the displacement is corrected by the displacement correcting function 1110e.

Blood flow data based on the Doppler effect of a target object in a scan range is extracted, by the Doppler processing function 1110i, by performing the frequency analysis on the reception signal for blood flow image generation stored by the signal storage function 1110c. In generation of blood flow data by the Doppler method, the reception signal data sequences of multiple frames are acquired in the temporal direction for the same measurement position. A moving target indicator (MTI) filter is applied to the reception signal data sequences by the Doppler processing function 1110i. Thus, information derived from a tissue in a still state across frames or a tissue with small movement (a clutter image component) is reduced, and information derived from blood flow (a blood-flow image component) is extracted. The processing circuit 110 calculates blood flow data, such as velocity, dispersion, and power, by the Doppler processing function 11101.

For the MTI filter, a filter in which the filter coefficient is fixed, such as a Butterworth-type infinite impulse response (IIR) filter and a polynomial regression filter, may be used. The MTI filter may also be an adaptive filter that varies the coefficient based on an input signal, using eigenvalue decomposition or singular value decomposition. Alternatively, the processing circuit 110 may decompose the reception signal into one or more bases using eigenvalue decomposition, singular value decomposition, or the like by the Doppler processing function 11101, to extract only a specific base and remove a clutter image component. Moreover, the processing circuit 110 may acquire a velocity vector for each coordinate in an image to determine a blood flow vector, which represent both a magnitude and a direction of blood flow using the vector Doppler method, the speckle tracking method, the vector flow mapping (VFM) method, or the like by the Doppler processing function 1110i. Besides the method explained herein, any method can be applied as long as it is possible to extract or enhance the information derived from blood flow included in the reception signal (measurement data), or to remove or reduce the information derived from a tissue.

The processing circuit 110 acquires data of multiple consecutive frames in the temporal direction that are acquired based on a result of ultrasonic scan on the subject by the acquiring function 110f as the first ultrasonic-image data. Moreover, as another example, the processing circuit 110 collects blood flow images (power Doppler images in this example) corresponding to multiple frames that are generated by the Doppler processing function 1110i, and acquires addition image data obtained by performing addition processing on these by the acquiring function 110f as the first ultrasonic-image data. The reception image or the measurement image can be handled as frame data, and one blood flow image obtained by summing multiple frames of this data may be used as the first ultrasonic-image data.

Furthermore, the processing circuit 110 performs processing of successively extracting image components representing the target object from the first ultrasonic-image data by the extracting function 1110g. Specifically, the processing circuit 110 applies a kernel to extract the consecutive components in a direction of interest to the first ultrasonic-image data that is acquired based on a result of the ultrasonic scan on the subject, to extract the second ultrasonic-image data by the extracting function 1110g. Moreover, the processing circuit 110 performs processing of outputting the second ultrasonic-image data based on an image component representing the target object successively extracted from the first ultrasonic-image data by the output function 1110h.

The image data output after performing the processing by the B-mode processing function 1110d, the Doppler processing function 1110i, and the extracting function 1110g is finally displayed on the display 135 after being subjected to the processing by the image generating function 111 illustrated in FIG. 9.

The processing circuit 110 that includes the image generating function 111 may be constituted of one or more processors and a memory. In this case, the respective functions illustrated in FIG. 10 are implemented by computer programs. For example, by reading and executing a program stored in the memory by a CPU, each function can be provided. The processing circuit 110 that includes an image generating function 111 may include a processor (a GPU, an FPGA, and the like) responsible for calculation of the B-mode processing function 1110d, the displacement correcting function 1110e, the Doppler processing function 11101, and the extracting function 1110g other than the CPU. The memory is preferable to include a non-volatile memory for storing programs, a memory for temporarily storing data such as reception signals, a working memory used by the CPU, and the like. Subsequently, a background according to the third embodiment will be explained.

As one method for super-resolution of blood flow signals without using a contrast agent, there has been a method in which a high-signal region that occur with low frequency in each frame is extracted for a blood flow signal that has passed through a wall filter, and signals with higher resolution than a resolution limit of ultrasonic waves are generated and accumulated across frames to obtain a blood flow signal with higher resolution than a conventional blood flow signal.

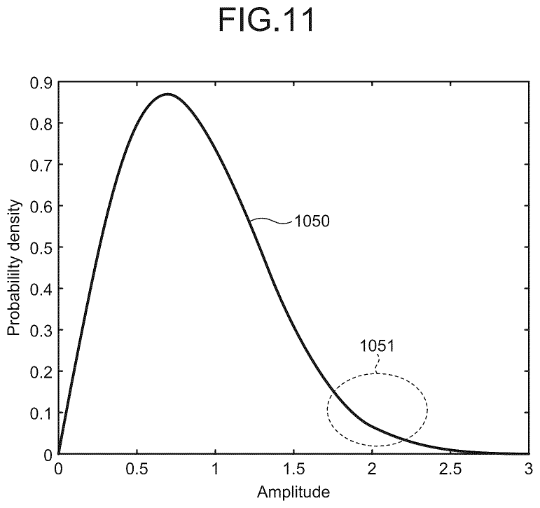

More specifically, for example, as illustrated in FIG. 11, it is described that an amplitude distribution characteristic 1050 of a speckle pattern in an ultrasonic image and a blood flow image can be closely approximated by a probability distribution called the Rayleigh distribution, and this fact is utilized, Thus, a signal that is high in brightness and spatially sparse, which is a portion in which an occurrence probability is small but an amplitude is high, for example, a signal corresponding to a region 1051 is extracted from each blood flow image, and is accumulated.

However, by this method, there has been a case in which the blood flow becomes discontinuous or not being rendered smoothly.

In view of this background, the image processing apparatus 100 as the medical information processing apparatus according to the third embodiment includes the processing circuit 110. The processing circuit 110 compares the pixel value at the region of interest and a pixel value at an adjacent position for each region of interest in each direction of interest for each of multiple regions of interest included in the first ultrasonic image data by the extracting function 1110g, and assigns weights to image components in the region of interest for each direction of interest.

Furthermore, the ultrasound diagnostic apparatus 10 according to the third embodiment includes transceiving circuit (the transmitting circuit 9 and the receiving circuit 11) as an executing unit, and the processing circuit 110. The executing unit causes the ultrasound probe 5 to perform ultrasonic scan on a subject. The processing circuit 110 extracts the second ultrasonic-image data by applying a kernel to extract the consecutive components in the direction of interest to the first ultrasonic-image data acquired based on a result of the ultrasonic scan by the extracting function 1110g.

Moreover, a program according to the third embodiment causes a computer to execute processing of extracting the second ultrasonic-image data by applying a kernel to extract consecutive components in the direction of interest to the first ultrasonic-image data that is acquired based on a result of ultrasonic scan to a subject.

Thus, it becomes possible to extract sequential target objects, and to perform imaging in which a target object, such as blood flow, is enhanced,

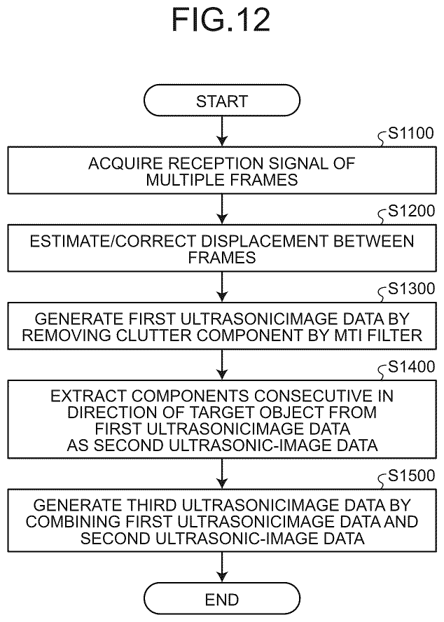

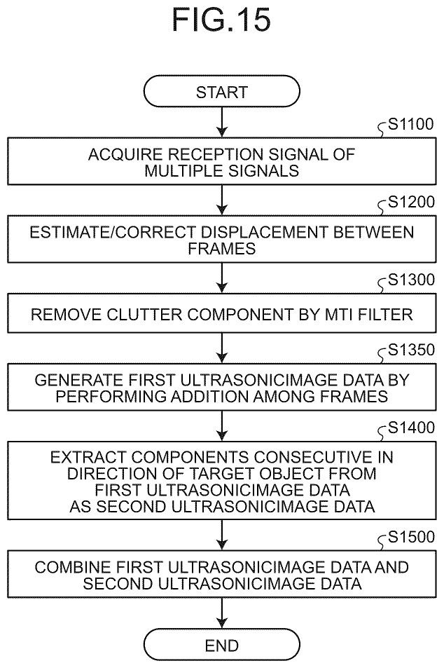

Processing performed by the image processing apparatus 100 as the medical information processing apparatus according to the third embodiment will be explained using FIG. 12 to FIG. 14. FIG. 12 is a flowchart explaining a flow of the processing performed by the image processing apparatus 100 according to the embodiment. Unlike a case of FIG. 15 in which kernel application processing is performed after the addition processing among frames to extract consecutive components, FIG. 12 illustrates a case in which kernel application processing is performed on each frame to extract consecutive components.

First, at step S1100, the processing circuit 110 collects multiple frames of data to be processed. That is, the processing circuit 110 acquires frame data 20 of multiple frames through scan on a subject performed by the ultrasound diagnostic apparatus 10, by the acquiring function 110f. That is, the processing circuit 110 collects multiple pieces of consecutive frame data in the temporal direction acquired by execution of ultrasonic scan, by the acquiring function 110f.

In other words, the processing circuit 110 sets a measurement position so as to include a region in which blood flow information is wished to be observed, and repeatedly performs transmission and reception of ultrasonic waves in a state in which the measurement position is fixed to the same position, to acquire a reception signal including a data sequence of multiple frames in the temporal direction by the acquiring function 110f. The number of frames of the reception signal when the super-resolution blood flow image is generated according to the present embodiment is generally larger than that when a normal Doppler image is generated. For example, while the number of frames (the number of packets) of the reception signal when a normal Doppler image is generated is approximately 5 to 20, the number of frames of the reception signal when the super-resolution blood flow image is generated is at least 100 frames or more, preferably on the order of several hundreds to several tens of thousands.

The frame rate of the ultrasound diagnostic apparatus 10 varies depending on the model and measurement method, but is approximately several hundred Hz to several thousand Hz. Therefore, measurement time of the reception signal for generating a super-resolution blood flow image is approximately several hundred milliseconds to several tens of seconds. The reception signal including a data sequence of multiple frames is subjected to the coherent addition and the quadrature detection processing by the coherent addition processing function 1110b, and is stored by the signal storage function 1110c.

Moreover, one frame in the data sequence may be the reception signal obtained by performing single ultrasound transmission/reception so as to include the observation region, or a frame obtained by summing multiple reception signals from repeated transmissions/receptions, so as to include the observation region. For example, plane waves or divergent waves are transmitted to include the observation region, and multiple reception signals with varying transmission angles are summed to form one frame of data.

Subsequently, at step S1200, the processing circuit 110 calculates an amount of displacement of a tissue occurred by body movement and the like between frames from the reception signal including the data sequence of multiple frames by the displacement correcting function 1110e. T The processing circuit 110 corrects the displacement by moving the reception signal such that position align in a frame in which integration processing described later is performed using the calculated displacement by the displacement correcting function 1110e. A reference frame when calculating the displacement may be a single frame for the entire data sequence, or the reference frame can be changed based on a position in the temporal direction (that is, multiple reference frames may be set), or the immediately preceding frame in the adjacent frames may be used as the reference frame. The reference frame can be arbitrarily selected from the data sequence and, for example, the first frame, a middle frame, or the last frame can be used as the reference frame. Subsequently, at step S1300, the processing circuit 110 reduces a clutter image component by applying the MTI filter to the reception signal including the data sequence of multiple frames in which the displacement has been corrected and extracts an image component derived from blood flow, to generate multiple frames of blood flow images by the Doppler processing function 1110i. In this case, for the design of the MTI filter, either a portion of the data sequence or the entire data sequence can be used.

Furthermore, parameters (cutoff frequency, the number of packets of an extracted frame and overlaps, and the like) of the MTI filter are preferable to be adjustable automatically or by a user according to a subject to be measured or the like. Thus, the processing circuit 110 generates the first ultrasonic-image data. In other words, by the processing from step S1100 to step S1300, the processing circuit 110 acquires the first ultrasonic-image data based on data of multiple frames consecutive in the temporal direction acquired based on a result of ultrasonic scan to the subject by the acquiring function 110f.

Subsequently, at step S1400, the processing circuit 110 applies a kernel to extract components consecutive in the direction of interest to the first ultrasonic-image data acquired based on a result of ultrasonic scan to the subject that is acquired by the processing up to step S1300, and extracts the second ultrasonic-image data obtained by extracting components consecutive in the direction representing the target object by the extracting function 1110g. In the embodiment in which the second ultrasonic-image data is extracted by applying a kernel to each frame as illustrated in FIG. 12, the processing circuit 110 applies a kernel to extract components consecutive in the direction of interest for each of the multiple frames to extract the second ultrasonic-image data by the extracting function 1110g.

In other words, the processing circuit 110 extracts an image component of a region of interest based on a comparison result between a pixel value of a position adjacent in the direction of interest for the region of interest and a pixel value of the region of interest for each of regions of interest included in the first ultrasonic-image data for each of the frames, as an image component representing the target object by the extracting function 1110g. The target object represents a structure of interest and includes, for example, at least one of blood, a body tissue, and a contrast agent. Furthermore, the direction representing the target object refers to a direction of blood flow, for example, when the target object is blood. That is, the processing circuit 110 generates the second ultrasonic-image data in which components consecutive in the direction of blood flow are extracted from the first ultrasonic-image data by the extracting function 1110g. Thus, the structure of the target object, for example, the structure of a blood vessel, can be rendered with a high image quality.

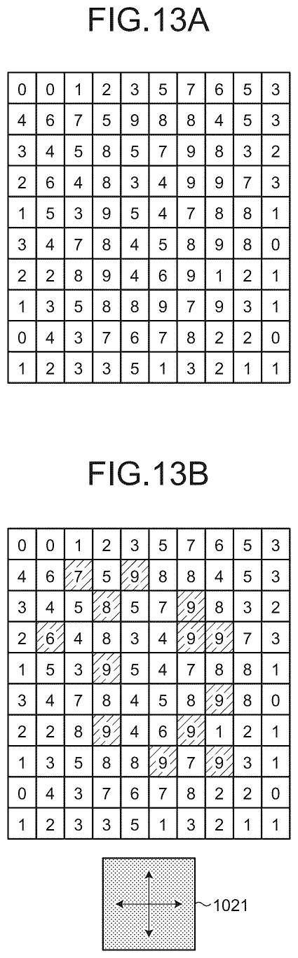

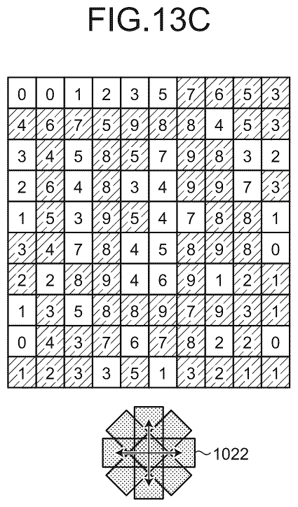

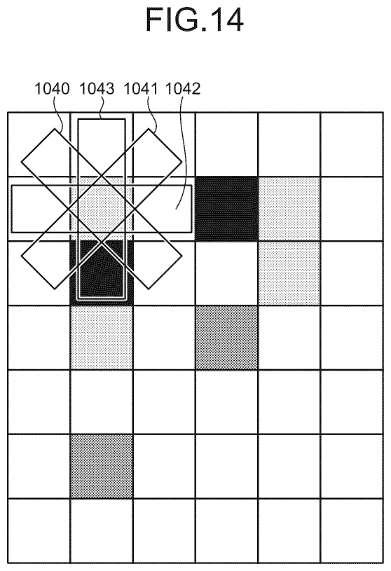

As a specific method of extracting components consecutive in the direction representing the target object, the processing circuit 110 extracts the second ultrasonic-image data by using a kernel extending in a specific direction. That is, at step S1400, the processing circuit 110 extracts the second ultrasonic-image data by applying the kernel extending in a specific direction to the first ultrasonic-image data by the extracting function 1110g. In FIG. 13C and FIG. 14C, a specific example of the kernel extending in a specific direction is illustrated,

Respective square cells in FIG. 13A represent pixels of the first ultrasonic-image data, and the number in each square indicates a signal value of each pixel. For example, when a high signal point is extracted from the first ultrasonic-image data, in a conventional method, for example, as illustrated in FIG. 13B, a high signal point is extracted using a kernel 1021 that spans nine squares of 3×3 including a point of interest and eight squares adjacent to the point of interest. In this case, the processing circuit 110 extracts, only when a point of interest has a higher signal value than any other signal points of the eight adjacent squared, the point of interest as a high signal point. As described, in the conventional method in which a high signal point is extracted using a kernel of 3×3, there has been a case in which a structure such as a blood vessel extending in a specific direction cannot be detected sufficiently. Accordingly, in the method according to the embodiment, the processing circuit 110 extracts the second ultrasonic-image data from the first ultrasonic-image data using a kernel extending in a specific direction, such as kernel 1040 and a kernel 1041 in a size of 3×1 as illustrated in FIG. 14, by the extracting function 1110g. The kernel 1040 is a kernel to compare a signal value of a point of interest with a signal value of a square diagonally one cell up and to the left, and a signal value of a square diagonally one cell down and to the right, and the processing circuit 110 extracts, when a signal value of one point is a larger signal value than a signal value of a square diagonally one cell up and to the left and a signal value of a square diagonally one cell down and to the right, the point as a high signal point in the second ultrasonic-image data using the kernel 1040 by the extracting function 1110g.

Moreover, the kernel 1041 is a kernel to compare a signal value of a point of interest with a signal value of a square diagonally one cell down and to the left, and a signal value of a square diagonally one cell up and to the right, and the processing circuit 110 extracts, when a signal value of one point is a larger signal value than a signal value of a square diagonally one cell down and to the left and a signal value of a square diagonally one cell up and to the right, the point as a high signal point in the second ultrasonic-image data using the kernel 1041 by the extracting function 1110g.

Furthermore, a kernel 1042 is a kernel to compare a signal value of a point of interest with a signal value of a square one cell left, and a signal value of a square one cell right, and the processing circuit 110 extracts, when a signal value of one point is a larger signal value than a signal value of a square one cell left and a signal value of a square one cell right, the point as a high signal point in the second ultrasonic-image data using the kernel 1042 by the extracting function 1110g.

Moreover, a kernel 1043 is a kernel to compare a signal value of a point of interest with a signal value of a square one cell above, and a signal value of a square one cell below, and the processing circuit 110 extracts, when a signal value of one point is a larger signal value than a signal value of a square one cell above and a signal value of a square one cell below, the point as a high signal point in the second ultrasonic-image data using the kernel 1043 by the extracting function 1110g.

That is, the processing circuit 110 extracts a pixel value of a point of interest as an image component representing the target object when the pixel value of the point of interest is larger than or equal to a pixel value of a position adjacent in one direction relative to the point of interest by the extracting function 1110g.

FIG. 13C illustrates an extraction result when the second ultrasonic-image data is extracted from the first ultrasonic-image data using the kernel 1022 extending in a specific direction as described. As illustrated in FIG. 14, when signal extraction is performed using a kernel that extends in a specific direction, such as the kernel 1040 and the kernel 1041, a high brightness signal region can be extracted in a relatively wide range compared to the case of FIG. 13B in which signal extraction is performed using a kernel extending in all adjacent directions.

As described, in the method according to the embodiment, the processing circuit 110 can extract components consecutive in the direction representing a target object appropriately by extracting the second ultrasonic-image data from the first ultrasonic-image data using, for example, a kernel of 3×1 that extends in a specific direction by the extracting function 1110g. That is, while extraction of image components representing a target object is discrete in the conventional method, extraction of image components representing a target object is continuous in the method of the embodiment. That is, in the embodiment, to enable continuous extraction of peak pixel values, extraction conditions can be more lenient compared to the conventional method. A region of interest for peak determination is basically in pixel unit, but may be a group unit having multiple pixels. When performing peak detection in a group unit, a pixel value for the group may be determined by using an average value of the pixel values included in the multiple groups, or by using an accumulation value.

As described above, the processing circuit 110 extracts an image component of a region of interest, for example, when a pixel value of the region of interest is larger than or equal to a pixel value at a position adjacent to the region of interest in one direction as an image component representing a target object by the extracting function 1110g. One direction refers to, for example, a direction 45 degrees to the lower left, a direction 45 degrees to the lower right, a direction 45 degrees to the upper left, a direction 45 degrees to the upper right, directions 45 degrees to the lower left and 45 degrees to the upper right, directions 45 degrees upper left and 45 degrees lower right, a left direction, a right direction, an upward direction, a downward direction, a horizontal direction, a vertical direction, and the like.

Moreover, the embodiment is not limited thereto, and the processing circuit 110 may extract an image component of a region of interest as an image component representing a target object, for example, when a pixel value of the region of interest is larger than or equal to a pixel value of a position adjacent to the region of interest in multiple directions out of the directions described above by the extracting function 1110g.

Furthermore, although a case in which a kernel of 3×1 is applied has been explained in the embodiment, the size of the kernel is not limited thereto, and the processing circuit 110 may extract an image component of a region of interest as an image component representing a target object by applying a kernel of other sizes, such as a kernel of 5×1 and a kernel of 7×1, by the extracting function 1110g.

In this case, the processing circuit 110 may extract an image component of a region of interest as an image component representing a target object, for example, when a pixel value of the region of interest is larger than or equal to pixel values of all positions extending in one direction out of pixels within a range to which the kernel is applied to the region of interest. Moreover, as another example, an image component of a region of interest can be extracted as an image component representing a target object when the number of pixels with pixel values larger than or equal to the pixel value of the region of interest exceeds a predetermined threshold in pixels extending in one direction out of pixels within a range to which the kernel is applied to the region of interest.

Furthermore, although a case in which a signal is extracted when a pixel value of a region of interest is larger than or equal to a pixel value of an adjacent position, the embodiment is not limited thereto, and a signal may be extracted when a pixel value of the region of interest is smaller than or equal to a pixel value of an adjacent position depending on a type and a format of data. That is, the processing circuit 110 may extract an image component of a region of interest as an image component representing a target object, for example, when a pixel value of the region of interest is smaller than or equal to a pixel value of a position adjacent to the region of interest in one direction relative to the region of interest by the extracting function 1110g.

Moreover, the embodiment is not limited thereto, and signal extraction may be omitted when a pixel value of a region of interest is equal to a pixel value of an adjacent position. In this case, the processing circuit 110 extracts an image component of a region of interest as an image component representing a target object, for example, only when a pixel value of the region of interest is larger than a pixel value of a position adjacent to the region of interest in one direction.

Furthermore, as another example, the processing circuit 110 may compare a pixel value with a pixel value of an adjacent position in each direction of interest relative to the region of interest, for each of regions of interests included in the first ultrasonic-image data, and may assign weights to image components of region of interests based on a comparison result of each direction of interest by the extracting function 1110g.

As an example, consider a case in which the processing circuit 110 performs signal extraction using four kernels of the kernels 1040, 1041, 1042, and 1043 illustrated in FIG. 14. In this case, the processing circuit 110 varies, for example, a weight of the kernels 1040 and 1041 in a diagonal direction and a weight of the kernels 1042 and 1043 in a horizontal direction. As an example, the processing circuit 110 sets the weight of the kernels 1040 and 1041 in a diagonal direction to “0.3”, and sets the weight of the kernels 1042 and 1043 in a horizontal direction to “0.2” by the extracting function 1110g.

Subsequently, the processing circuit 110 performs comparison between a pixel value of a region of interest and a pixel value of an adjacent position for each direction of interest, and acquires a comparison result of each direction of interest by the extracting function 1110g. For example, the processing circuit 110 acquires, by the extracting function 1110g, “1” when a pixel value of a region of interest and a pixel value of an adjacent position satisfy a predetermined condition in a direction of interest, and acquires “0” when the predetermined condition is not satisfied. Thereafter, the processing circuit 110 performs weighted addition on a result acquired in each direction of interest by the extracting function 1110g. For example, when the processing circuit 110 acquires an evaluation value “1” for the directions of the kernels 1040 and 1043, and acquires an evaluation value “0” for the directions of the kernels 1041 and 1042 by the extracting function 1110g, the processing circuit 110 performs weighted addition on the evaluation values, to acquire the weighted addition value as 1×0,3+0×0.3+0×0.2+1×0.2=0.5.

Finally, the processing circuit 110 extracts, for example, when the acquired weighted addition value is compared with a threshold, and it is equal to or higher than the threshold, extracts the signal as a high signal point by the extracting function 1110g. For example, when the threshold is “0.5”, the processing circuit 110 extracts the signal as a high signal point by the extracting function 1110g.

As another example, the processing circuit 110 may perform comparison between a pixel value of a region of interest and a pixel value of an adjacent position for each direction of interest relative to the region of interest for each of multiple region of interests included in the first ultrasonic-image data, and may assign a weight to an image component of the region of interest for each direction of interest by the extracting function 1110g.

The embodiment is not limited to the example described above, and the processing circuit 110 may extract the second ultrasonic-image data from the first ultrasonic-image data using a trained model using artificiall intelligence (AI) and the like by the extracting function 1110g. That is, through input of ultrasonic image data, the processing circuit 110 may input the first ultrasonic-image data to a trained model to successively extract image components representing a target object included in the ultrasonic image data, thereby acquiring the image components representing the target object successively extracted from the trained model by the extracting function 1110g.

As an example, the processing circuit 110 extracts the second ultrasonic-image data from the first ultrasonic-image data using a neural network by the extracting function 1110g. As an example, the processing circuit 110 conducts training of the neural network, for example, using image data before extraction of a blood vessel is performed as input data, and image data that has been subjected to blood-vessel extraction processing by another method as output data. Moreover, as another example, the processing circuit 110 extracts image data subjected to the blood-vessel extraction processing as the second ultrasonic-image data, from image data before blood vessel extraction by machine learning using a clustering algorithm.

As above, the processing at step S1400 has been explained, and in the embodiment illustrated in FIG. 12, the processing circuit 110 applies the extraction processing described above in each frame by the extracting function 1110g. That is, the processing circuit 110 extracts the second ultrasonic-image data by applying a kernel to extract a high signal point for each of multiple frames by the extracting function 1110g.

Referring back to FIG. 12, at step S1500, the processing circuit 110 generates the third ultrasonic-image data based on the first ultrasonic-image data generated at step S1300 and the second ultrasonic-image data extracted at step S1400 by the output function 1110h. As an example, the processing circuit 110 generates the third ultrasonic-image data by combining the first ultrasonic-image data and the second ultrasonic-image data by the output function 1110h. As an example, the processing circuit 110 may output composite image data that is obtained by subjecting an image component representing a target object successively extracted and the first ultrasonic-image data to composition processing, as the second ultrasonic-image data.

Specifically, the processing circuit 110 performs weighted addition on the first ultrasonic-image data and the second ultrasonic-image data to generate the third ultrasonic-image data by the output function 1110h, For example, the processing circuit 110 sets a weight of the first ultrasonic-image data to 0.8, and sets a weight of the second ultrasonic-image data to 0.2, and performs weighted addition on the first ultrasonic-image data and the second ultrasonic-image data, to generate the third ultrasonic-image data by the output function 1110h. Moreover, for example, when extraction of the second ultrasonic-image data is performed in multiple directions at step S1400, the processing circuit 110 may perform weighted addition while assigning various weights according to the direction of the extracted second ultrasonic-image data to generate the third ultrasonic-image data by the output function 1110h,

Moreover, at step S1500, the processing circuit 110 may perform composition after normalizing the first ultrasonic-image data and the second ultrasonic-image data, to generate the third ultrasonic-image data by the output function 1110h. In other words, the processing circuit 110 may perform composition after performing signal processing on each of signal data such that maximum values or average values of a signal value become equivalent between the first ultrasonic-image data and the second ultrasonic-image data, to generate the third ultrasonic-image data by the output function 1110h.

Furthermore, as another example, the processing circuit 110 may correct the first ultrasonic-image data using the second ultrasonic-image data, to generate the third ultrasonic-image data obtained by correcting the first ultrasonic-image data by the output function 1110h. That is, the processing circuit 110 may output correction image data that is obtained by subjecting the first ultrasonic-image data to correction processing based on image components representing a target object successively extracted, as the second ultrasonic-image data by the output function 1110h.

As an example, the processing circuit 110 may replace data of a high signal point in which the second ultrasonic-image data is present out of the first ultrasonic-image data with the second ultrasonic-image data, to generate the third ultrasonic-image data, which is data obtained by correcting the first ultrasonic-image data, by the output function 1110h.

The embodiment is not limited to the example described above, and the processing circuit 110 may additionally perform various kinds of processing on the second ultrasonic-image data or the third ultrasonic-image data by the output function 1110h.

As an example, the processing circuit 110 may perform spatial filtering processing such as noise removal, or apply a smoothing filter in a spatial or temporal direction on the second ultrasonic-image data generated at step S1400, and may generate the third ultrasonic-image data using signal data on which the spatial filtering processing has been performed or to which the smoothing filter in the spatial or temporal direction has been applied at step S1500, by the output function 1110h. Furthermore, as another example, the processing circuit 110 may generate the third ultrasonic-image data using the signal data subjected to the spatial filtering processing at step S1500, after performing processing in the frame direction, for example, operation of replacing signal data with a moving average value of predetermined number of frames, on the second ultrasonic-image data generated at step S1400, by the output function 1110h. Moreover, the processing circuit 110 may perform the spatial filtering processing or the processing in the frame direction using AI by the output function 1110h. As an example, the processing circuit 110 may perform the spatial filtering processing on the second ultrasonic-image data based on a neural network that has been trained using a low quality image as input data and a high quality image as output data by the output function 1110h.

Furthermore, as another example, these spatial filtering processing and processing in the frame direction may be performed on the generated third ultrasonic-image data. That is, the processing circuit 110 may perform the spatial filtering processing such as noise removal or the processing in the frame direction, for example, the operation of replacing signal data with a moving average of a predetermined number of frames, on the third ultrasonic-image data generated at step S1400 by the output function 1110h. Moreover, the processing circuit 110 may perform the spatial filtering processing or the processing in the frame direction on the third ultrasonic-image data using AI by the output function 1110h.

Furthermore, as another example, the processing circuit 110 may output image data including an image component representing a target object successively extracted as the second ultrasonic-image data by the output function 1110h.

The processing circuit 110 causes the display 135 to display an ultrasonic image data based on the generated third ultrasonic-image data by the display control function 114. Moreover, the processing circuit 110 may cause the display 135 to display the second ultrasonic-image data by the display control function 114.

In the example in FIG. 12, a case in which the processing circuit 110 performs processing of extracting a high signal point per frame by the extracting function 1110g at step S1400 has been explained. However, the embodiment is not limited thereto. That is, the processing circuit 110 may perform the kernel application processing at step S1400 on data that has subjected to the frame addition processing by the extracting function 1110g.

FIG. 15 illustrates a flowchart of the embodiment. A flow of processing illustrated in FIG. 15 is the same as that of FIG. 12 except a point that processing at step S1350 is added and, therefore, duplicated explanation of the processing other than step S1350 will be omitted.

At step S1350, the processing circuit 110 acquires addition image data that is obtained by performing subjecting multiple pieces of frame data consecutive in the temporal direction acquired and collected through ultrasonic scan as the first ultrasonic-image data by the extracting function 1110g. At step S1400, the processing circuit 110 performs the kernel application processing described above on the first ultrasonic-image data, which is data subjected to the addition processing, and performs the same processing thereafter by the extracting function 1110g. Thus, even when the signal intensity of each frame is insufficient, the quality of an output image can be improved by performing frame addition to enhance the signal intensity,

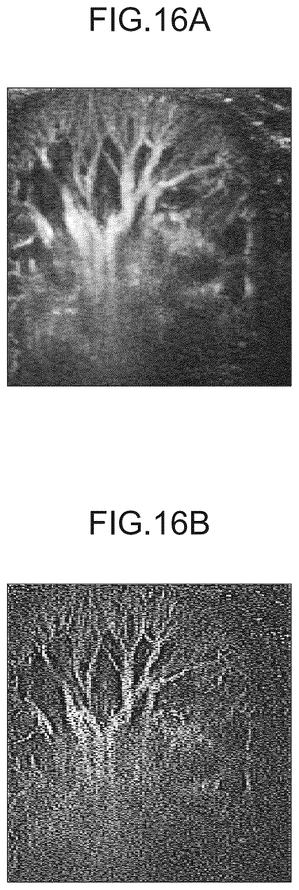

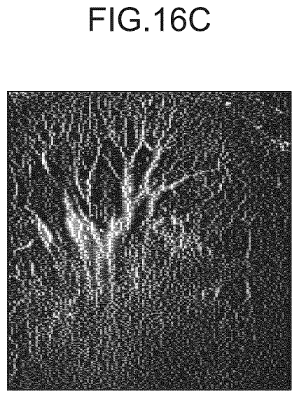

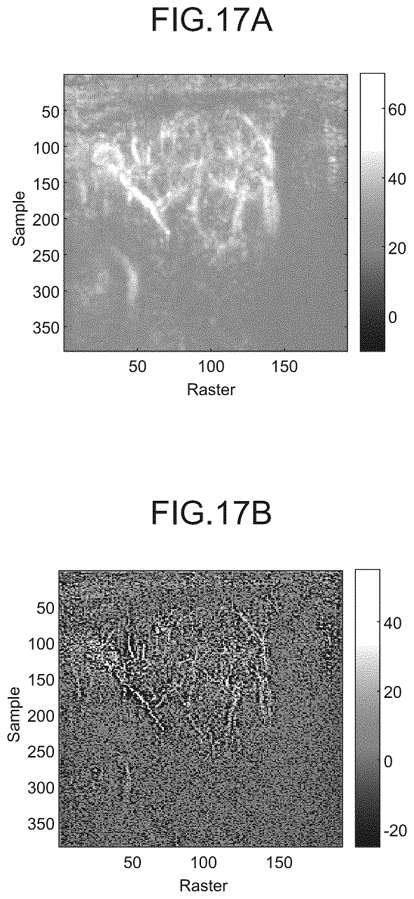

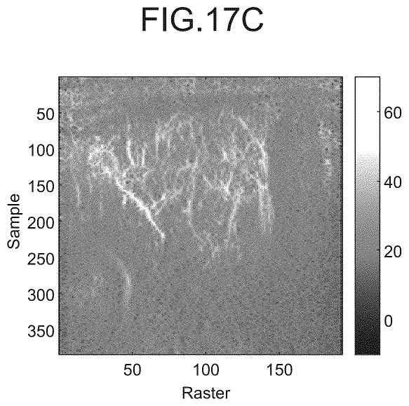

An ultrasonic image 29 output by the method according to the embodiment will be explained using FIG. 16A to FIG. 17C. FIG. 16A and FIG. 17A are a first comparative example, and illustrate an ultrasonic image that is obtained by using the super micro-vascular imaging (SMI) method. FIG. 16B and FIG. 17B are a second comparative example, and illustrate an ultrasonic image that is obtained by using the super-resolution imaging method explained in FIG. 11. FIG. 16C and FIG. 17C illustrate an ultrasonic image that is output by the method according to the embodiment. In the method according to the embodiment, blood flow can be rendered at high resolution compared to the SMI method, which is the first comparative example, and peripheral blood flow can be rendered in detail and with continuously.

As described above, the image processing apparatus 100 according to the embodiment can improve the visibility of a target object, being capable of further enhancing minute blood flow and the like.

The embodiment is not limited thereto.

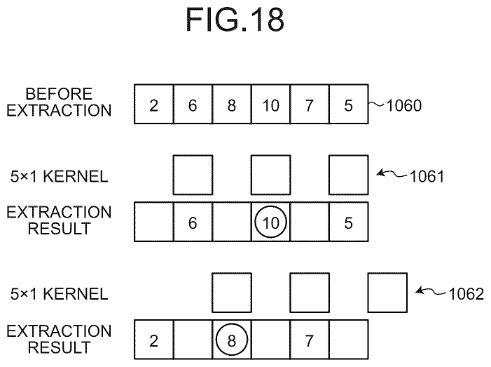

As for the kernel for peak detection according to the embodiment, a case in which a kernel extending in the size of 3×1 in a predetermined direction is used has been explained, but the embodiment is not limited to the example described above. As the kernel using peak detection, for example, as illustrated in a kernel 1061 in FIG. 18, for example, a kernel of 5×1 and that is a filter to extract only the first, the third, and the fifth cells from the left out of 5×1 cells may be used as the kernel, and by comparing a signal value extracted thereafter with an adjacent signal value, signal extraction may be performed. That is, as the kernel used for the peak detection according to the embodiment, a kernel that acts on pixels other than adjacent pixels can also be used. When the kernel 1061 as described is applied to, for example, a pre-extraction signal 1060, a signal having a signal value “10” is extracted as illustrated in FIG. 18. Moreover, when a kernel 1062 that is obtained by shifting the kernel 1061 as described is applied to the pre-extraction signal 1060, a signal having a signal value “8” is extracted. Therefore, by using such a kernel, signals having a signal value “8” and a signal with “10” can be extracted, and the peak extraction can be performed more flexibly compared to the conventional method. In other words, while extraction of image components representing a target object is discrete in the conventional method, extraction of image components representing a target object is continuous in the method of the embodiment.

Moreover, at step S1300, a type of data of the first ultrasonic-image data acquired by the processing circuit 110 using the acquiring function 110f can be various data other than, for example, the Doppler image data. As an example, at step S1300, the processing circuit 110 may acquire B-mode image data as the first ultrasonic-image data by the acquiring function 110f. Because the B-mode image data has a characteristic that a signal value becomes large at an edge portion of an organ, by acquiring the B-mode image data as the first ultrasonic-image data to perform processing, the edge portion of an organ can be rendered in enhanced manner. Furthermore, in the embodiment, a case in which the frame data acquired by the processing circuit 110 using the acquiring function 110f is a blood flow image has been explained, but the embodiment is not limited thereto. The frame data acquired by the processing circuit 110 using the acquiring function 110f may be the reception signal, the measurement signal after DAS, or the blood flow signal after clutter removal. In this case, the processing circuit 110 may acquire one blood flow image that is obtained using addition of multiple frames of the reception image/the measurement image, and the like by the acquiring function 110f as first ultrasonic-image data.

Furthermore, at step S1100, the processing circuit 110 may acquire data for each frequency, instead of data for each frame by the acquiring function 110f. In this case, at step S1200, the processing circuit 110 adds data for each frequency instead of performing the addition processing for a frame, to acquire the first ultrasonic-image data by the acquiring function 110f.

Moreover, the first ultrasonic-image data may be data to which the smoothing filter is applied. That is, the processing circuit 110 may acquire data of multiple frames consecutive in the temporal direction that is acquired based on a result of ultrasonic scan on a subject, to which the smoothing filter is applied in the spatial direction or the temporal direction as the first ultrasonic-image data by the acquiring function 110f.

### Fourth Embodiment

Subsequently, a fourth embodiment will be explained. A configuration of the ultrasound diagnostic apparatus 10 according to the fourth embodiment is the same as the configuration illustrated in FIG. 9 in the third embodiment except the processing circuit and, therefore, duplicated explanation will be omitted.

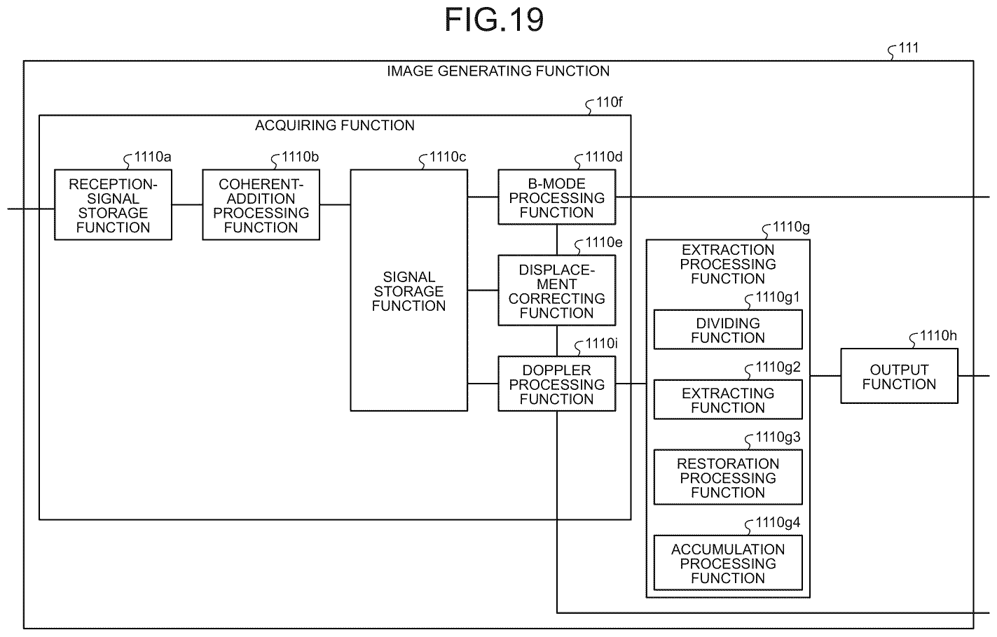

FIG. 19 illustrates a configuration of the image generating function 111 included in the processing circuit 110 in the fourth embodiment. More specifically, the image generating function 111 includes the acquiring function 110f, an extraction processing function 1110g, and an output function 1110h. More specifically, the acquiring function 110f is constituted of the reception-signal storage function 1110a, the coherent-addition processing function 110b, the signal storage function 1110c, the B-mode processing function 1110d, the displacement correcting function 1110e, the Doppler processing function 11101, and the like. The extraction processing function 1110g is constituted of a dividing function 1110g1, an extracting function 1110g2, a restoration processing function 1110g3, and an accumulation processing function 1110g4. In the embodiment, respective processing functions performed by the reception-signal storage function 1110a, the coherent-addition processing function 1110b, the signal storage function 1110c, the B-mode processing function 1110d, the displacement correcting function 1110e, the acquiring function 110f, the extraction processing function 1110g, the dividing function 1110g1, the extracting function 1110g2, the restoration processing function 1110g3, the accumulation processing function 1110g4, the output function 1110h, the Doppler processing function 1110i, the image generating function 111, the image processing function 112, the control function 113, the display control function 114, and the like and a trained model are stored in the memory 132 in a form of computer-executable program. The processing circuit 110 is a processor that reads out and executes a program from the memory 132, and thereby implements a function corresponding to each program. In other words, the processing circuit 110 that has read out the respective programs is to have the respective functions indicated in the processing circuit 110 in FIG. 9, or in the image generating function 111 in FIG. 19.

In FIG. 9 and FIG. 19, the image generating function 111, the image processing function 112, the control function 113, the display control function 114, the acquiring function 110f, the extraction processing function 1110g, the dividing function 1110g1, the extracting function 1110g2, the restoration processing function 1110g3, the accumulation processing function 1110g4, and the output function 1110h are one example of an image generating unit, a processing unit, a control unit, a display unit, an acquiring unit, an extraction processing unit, a dividing unit, an extracting unit, a restoration processing unit, an accumulation processing unit, and an output unit, respectively.

### Configuration of Reception-Signal Processing Block

FIG. 19 is a block diagram illustrating an example of the image generating function 111. The image generating function 111 is constituted of the acquiring function 110f, the extraction processing function 1110g, and the output function 1110h. Moreover, the acquiring function 110f is constituted of the reception-signal storage function 1110a, the coherent-addition processing function 1110b, the signal storage function 1110c, the B-mode processing function 1110d, the displacement correcting function 1110e, and the Doppler processing function 1110i. The extraction processing function 1110g is constituted of the dividing function 1110g1, the extracting function 1110g2, the restoration processing function 1110g3, and the accumulation processing function 1110g4. The reception-signal storage function 1110a stores the reception signal received by the receiving circuit 11. Depending on a type of an apparatus configuration or a reception signal, it may be configured such that the reception signal received by the receiving circuit is not to be stored in the reception-signal storage function 1110a, but stored in the signal storage function 1110c after the coherent-addition processing function 1110b described later. Moreover, the reception-signal storage function 1110a is constituted of a block common to the signal storage function 1110c, and may store the reception signal from the receiving circuit 11 and the reception signal after the coherent-addition processing function 1110b.

The coherent-addition processing function 1110b performs the coherent addition and the quadrature detection processing on the reception signal acquired by the receiving circuit 11, and stores the reception signal subjected to the processing in the signal storage function 1110c. The coherent addition processing is processing to form a reception ultrasound beam by varying the delay time and weight for each transducer 101, and by combining the reception signals from multiple transducers 101, and is called delay and DAS beam forming also. The quadrature detection processing is processing to convert the reception signal into a baseband in-phase signal (I signal) and a quadrature-phase signal (Q signal). The coherent addition processing and the quadrature detection processing are performed based on a device arrangement input by the control function 113 and various conditions (aperture control, signal filtering) for image generation. The reception signal after subjected to the coherent addition processing and the quadrature detection processing is stored by the signal storage function 1110c. Although an example of typical DAS beamforming is described herein, any type of processing for forming a reception ultrasound beam, such as adaptive beamforming, model-based processing, or processing using machine learning, may be used.

The reception signal (digital data) acquired by the receiving circuit 11 is also called RAW data. The reception signal (digital data) acquired by the coherent-addition processing function 1110b is also called RF data. The RAW data and the RF data are one example of measurement data acquired based on a reception signal of an ultrasonic wave reflected in a living body.

The B-mode processing function 1110d generates image data in which a signal intensity is expressed by brightness intensity at each point in an observation region by performing envelope detection processing, logarithmic compression processing, and the like on the reception signal for B-mode image generation stored by the signal storage function 1110c. Moreover, the B-mode processing function 1110d may perform the B-mode processing on the reception signal in which the displacement is corrected by the displacement correcting function 1110e.

The displacement correcting function 1110e calculates an amount of displacement of a tissue due to body movement among frames from the reception signals acquired from multiple frames. Generally, it is calculated using block-matching operations called speckle tracking method. By setting a region of interest within a frame, and by tracking the region of interest across frames using correlation, the displacement of the region of interest is calculated. By setting multiple regions of interest within a frame, the displacement for the entire frame is calculated. Although an example using correlation between frames is described herein, any method that can determine the displacement of the region of interest can be used. In the ultrasonic wave signal, because tracking of speckle, which is the scattered image of ultrasonic wave reflected from a scatterer in a body tissue, is performed, it is referred to as speckle tracking. Correlation calculations of the region of interest between frames can be performed on the reception signals after any one of the coherent addition processing, the quadrature detection processing, and the envelope detection processing. Moreover, the correlation calculation may be performed on a time waveform of the reception signal or on a frequency-domain data obtained by performing a discrete Fourier transform on the reception signal. The displacement correcting function 1110e corrects the displacement by moving the reception signal with respect to a reference frame using a calculated displacement. The displacement to be corrected may be set as a uniform value for the entire frame, such as an average displacement of each region of interest, or it may vary within the frame for each region of interest. Furthermore, the displacement of the entire frame calculated for each region of interest may be interpolated in a frame or in a temporal direction of data sequence using linear interpolation, spline interpolation, or the like. Moreover, when correcting the displacement, to correct more precisely, the reception signal may be moved similarly after interpolation.

The Doppler processing function 1110i extracts blood flow data from the reception signal for blood flow image generation stored by the signal storage function 100c. Moreover, the Doppler processing may be performed, by the Doppler processing function 11101, with respect to the reception signal in which the displacement is corrected by the displacement correcting function 1110e.

Blood flow data based on the Doppler effect of a target object in a scan range is extracted, by the Doppler processing function 1110i, by performing the frequency analysis on the reception signal for blood flow image generation stored by the signal storage function 1110c.

In generation of blood flow data by the Doppler method, the reception signal data sequences of multiple frames are acquired in the temporal direction for the same measurement position. An MTI filter is applied to the reception signal data sequences by the Doppler processing function 11101. Thus, information derived from a tissue in a still state across frames or a tissue with small movement (a clutter image component) is reduced, and information derived from blood flow (a blood flow component) is extracted. The processing circuit 110 calculates blood flow data, such as velocity, dispersion, and power, from this blood-flow image component by the Doppler processing function 1110i.

For the MTI filter, a filter in which the filter coefficient is fixed, such as a Butterworth-type IIR filter and a polynomial regression filter, may be used. The MTI filter may also be an adaptive filter that varies the coefficient based on an input signal, using eigenvalue decomposition or singular value decomposition.

Alternatively, the processing circuit 110 may decompose the reception signal into one or more bases using eigenvalue decomposition, singular value decomposition, or the like by the Doppler processing function 1110i, to extract only a specific base and remove a clutter image component. Moreover, the processing circuit 110 may acquire a velocity vector for each coordinate in an image to determine a blood flow vector, which represents both a magnitude and a direction of blood flow using the vector Doppler method, the speckle tracking method, the vector flow mapping method, or the like by the Doppler processing function 1110i. Besides the method explained herein, any method can be applied as long as it is possible to extract or enhance the information derived from blood flow included in the reception signal (measurement data), or to remove or reduce the information derived from a tissue.

The processing circuit 110 acquires data of multiple consecutive frames in the temporal direction that are acquired based on a result of ultrasonic scan on the subject by the acquiring function 110f as the first ultrasonic-image data. Moreover, as another example, the processing circuit 110 collects blood flow images (power Doppler images in this example) corresponding to multiple frames that are generated by the Doppler processing function 1110i, and acquires addition image data obtained by performing addition processing on these by the acquiring function 110f as the first ultrasonic-image data. The reception image or the measurement image can be handled as frame data, and one blood flow image obtained by summing multiple frames of this data may be used as the first ultrasonic-image data.

Furthermore, the processing circuit 110 performs processing of successively extracting image components representing the target object from the first ultrasonic-image data by the extracting function 1110g. Details of the extraction processing function 1110g will be described later. Moreover, the processing circuit 110 performs processing of outputting the second ultrasonic-image data based on an image component representing the target object successively extracted from the first ultrasonic-image data by the output function 1110h.

The image data output after performing the processing by the B-mode processing function 1110d, the Doppler processing function 1110i, and the extracting function 1110g is finally displayed on the display 135 after being subjected to the processing by the image generating function 111 illustrated in FIG. 9.

The processing circuit 110 that includes the image generating function 111 may be constituted of one or more processors and a memory. In this case, the respective functions illustrated in FIG. 19 are implemented by computer programs. For example, by reading and executing a program stored in the memory by a CPU, each function can be provided. The processing circuit 110 that includes an image generating function 111 may include a processor (a GPU, an FPGA, and the like) responsible for calculation of the B-mode processing function 1110d, the displacement correction function 1110e, the Doppler processing function 11101, and the extracting function 1110g other than the CPU. The memory is preferable to include a non-volatile memory for storing programs, a memory for temporarily storing data such as reception signals, a working memory used by the CPU, and the like.

Subsequently, a background according to the fourth embodiment will be explained. As has already been explained in the third embodiment, as one method for super-resolution of blood flow signals without using a contrast agent, there has been a method in which a high-signal region that occur with low frequency in each frame is extracted for a blood flow signal that has passed through a wall filter, and signals with higher resolution than a resolution limit of ultrasonic waves are generated and accumulated across frames to obtain a blood flow signal with higher resolution than a conventional blood flow signal.

However, by this method, there has been a case in which the blood flow becomes discontinuous or not being rendered smoothly.

In view of this background, the image processing apparatus 100 as the medical information processing apparatus according to the embodiment includes the processing circuit 110. The processing circuit 110 divides the first ultrasonic-image data into multiple pieces of the second ultrasonic-image data by the dividing function 1110g1. The processing circuit 110 extracts the third ultrasonic-image data by performing peak extraction from each of the second ultrasonic-image data by the extracting function 1110g2. The processing circuit 110 generates fourth ultrasonic-image data by allocating the third ultrasonic-image data to a corresponding position on the first ultrasonic-image data by the restoration processing function 1110g3. The processing circuit 110 generates fifth ultrasonic-image data by integrating the fourth ultrasonic-image data by the accumulation processing function 1110g4.

Moreover, the ultrasound diagnostic apparatus 10 according to the embodiment includes a transceiving circuit (the transmitting circuit 9 and the receiving circuit 11) as an executing unit, and the processing circuit 110. The executing unit causes the ultrasound probe 5 to perform ultrasonic scan on a subject. The processing circuit 110 divides the first ultrasonic-image data int multiple pieces of the second ultrasonic-image data by the dividing function 1110g1. The processing circuit 110 performs peak extraction to extract the third ultrasonic-image data from each of the second ultrasonic-image data by the extracting function 1110g2. The processing circuit 110 allocates the third ultrasonic-image data to a corresponding position on the first ultrasonic-image data to generate the fourth ultrasonic-image data by the restoration processing function 1110g3. The processing circuit 110 generates fifth ultrasonic-image data by integrating the fourth ultrasonic-image data by the accumulation processing function 1110g4. Furthermore, a program according to the embodiment cause a computer to execute processing of dividing the first ultrasonic-image data into multiple pieces of the second ultrasonic-image data, extracting the third ultrasonic-image data by performing peak extraction from each of the second ultrasonic-image data, generating the fourth ultrasonic-image data by allocating the third ultrasonic-image data to a corresponding position on the first ultrasonic-image data, and generating fifth ultrasonic-image data by integrating the fourth ultrasonic-image data.

Thus, it becomes possible to extract sequential target objects, and to perform imaging in which a target object, such as blood flow, is enhanced.

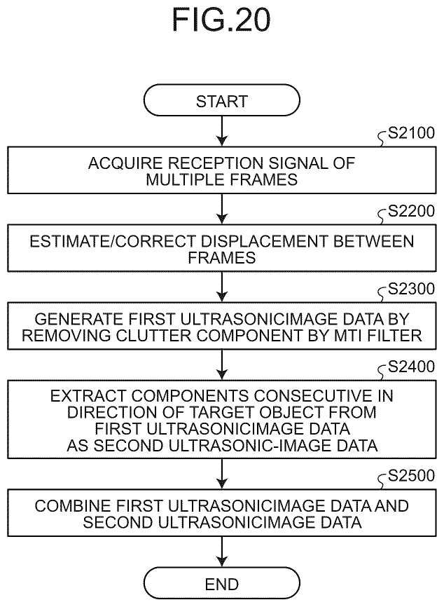

Processing performed by the image processing apparatus 100 as the medical information processing apparatus according to the embodiment will be explained using FIG. 20 to FIG. 24. FIG. 20 is a flowchart explaining a flow of the processing performed by the image processing apparatus 100 according to the embodiment.

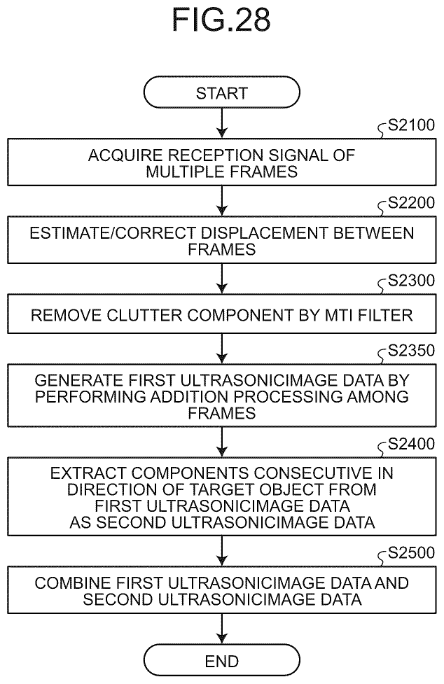

Unlike a case of FIG. 28 in which processing of extracting consecutive components after the addition processing is performed across frames, FIG. 20 illustrates a case in which extraction processing is performed for each frame to extract consecutive components.

First, at step S2100, the processing circuit 110 collects multiple frames of data to be processed, That is, the processing circuit 110 acquires frame data of multiple frames through scan on a subject performed by the ultrasound diagnostic apparatus 10, by the acquiring function 110f. That is, the processing circuit 110 collects multiple pieces of consecutive frame data in the temporal direction acquired by execution of ultrasonic scan, by the acquiring function 110f.

In other words, the processing circuit 110 sets a measurement position so as to include a region in which blood flow information is wished to be observed, and repeatedly performs transmission and reception of ultrasonic waves in a state in which the measurement position is fixed to the same position, to acquire a reception signal including a data sequence of multiple frames in the temporal direction by the acquiring function 110f. The number of frames of the reception signal when the super-resolution blood flow image is generated according to the present embodiment is generally larger than that when a normal Doppler image is generated. For example, while the number of frames (the number of packets) of the reception signal when a normal Doppler image is generated is approximately 5 to 20, the number of frames of the reception signal when the super-resolution blood flow image is generated is at least 100 frames or more, preferably on the order of several hundreds to several tens of thousands.

The frame rate of the ultrasound diagnostic apparatus 10 varies depending on the model and measurement method, but is approximately several hundred Hz to several thousand Hz. Therefore, measurement time of the reception signal for generating a super-resolution blood flow image is approximately several hundred milliseconds to several tens of seconds. The reception signal including a data sequence of multiple frames are subjected to the coherent addition and the quadrature detection processing via the coherent addition processing function 1110b, and is stored by the signal storage function 1110c. The reception signal including a data sequence of multiple frames is subjected to the coherent addition and the quadrature detection processing via the coherent-addition processing function 1110b, and is stored by the signal storage function 1110c.

Moreover, one frame in the data sequence may be the reception signal obtained by performing single ultrasound transmission/reception so as to include the observation region, or a frame obtained by summing multiple reception signals from repeated transmissions/receptions, so as to include the observation region. For example, plane waves or divergent waves are transmitted to include the observation region, and multiple reception signals with varying transmission angles are summed to form one frame of data.

Subsequently, at step S2200, the processing circuit 110 calculates an amount of displacement of a tissue occurred by body movement and the like between frames from the reception signal including the data sequence of multiple frames by the displacement correcting function 1110e. The processing circuit 110 corrects the displacement by moving the reception signal such that position align in a frame in which integration processing described later is performed using the calculated displacement by the displacement correcting function 1110e.

A reference frame when calculating the displacement may be a single frame for the entire data sequence, or the reference frame can be changed based on a position in the temporal direction (that is, multiple reference frames may be set), or the immediately preceding frame in the adjacent frames may be used as the reference frame. The reference frame can be arbitrarily selected from the data sequence and, for example, the first frame, a middle frame, or the last frame can be used as the reference frame.

Subsequently, at step S2300, the processing circuit 110 reduces a clutter image component by applying the MTI filter to the reception signal including the data sequence of multiple frames in which the displacement has been corrected and extracts an image component derived from blood flow, to generate multiple frames of blood flow images by the Doppler processing function 1110i. In this case, for the design of the MTI filter, either a portion of the data sequence or the entire data sequence can be used.

Furthermore, parameters (cutoff frequency, the number of packets of an extracted frame and overlaps, and the like) of the MTI filter are preferable to be adjustable automatically or by a user according to a type of a reception signal, a subject to be measured, or the like. Thus, the processing circuit 110 generates the first ultrasonic-image data. In other words, by the processing from step S2100 to step S2300, the processing circuit 110 acquires the first ultrasonic-image data based on data of multiple frames consecutive in the temporal direction acquired based on a result of ultrasonic scan to the subject by the acquiring function 110f.

Subsequently, at step S2400, the processing circuit 110 extracts the fifth ultrasonic-image data in which components consecutive in the direction representing the target object from the first ultrasonic-image data obtained based on a result of ultrasonic scan on a subject obtained by the processing up to step S2300 by the extraction function 1110g. In the embodiment in which the fifth ultrasonic-image data is extracted by applying the extraction processing of a high signal point per frame as illustrated in FIG. 20, the processing circuit 110 applies a kernel to extract a high signal point for each of the multiple frames to extract the fifth ultrasonic-image data by the extraction function 1110g.

In other words, the processing circuit 110 extracts an image component of a point of interest as an image component representing the target object for each of multiple points of interest included in the first ultrasonic-image data for each of the frames, by the extraction function 1110g. The target object represents a structure of interest and includes, for example, at least one of blood, a body tissue, and a contrast agent. Furthermore, the direction representing the target object refers to a direction of blood flow, for example, when the target object is blood. That is, the processing circuit 110 generates the fifth ultrasonic-image data in which components consecutive in the direction of blood flow are extracted from the first ultrasonic-image data by the extraction processing function 1110g. Thus, the structure of the target object, for example, the structure of a blood vessel, can be rendered with high image quality.

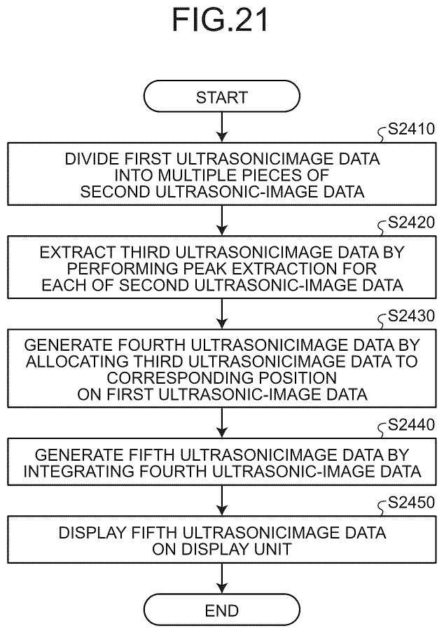

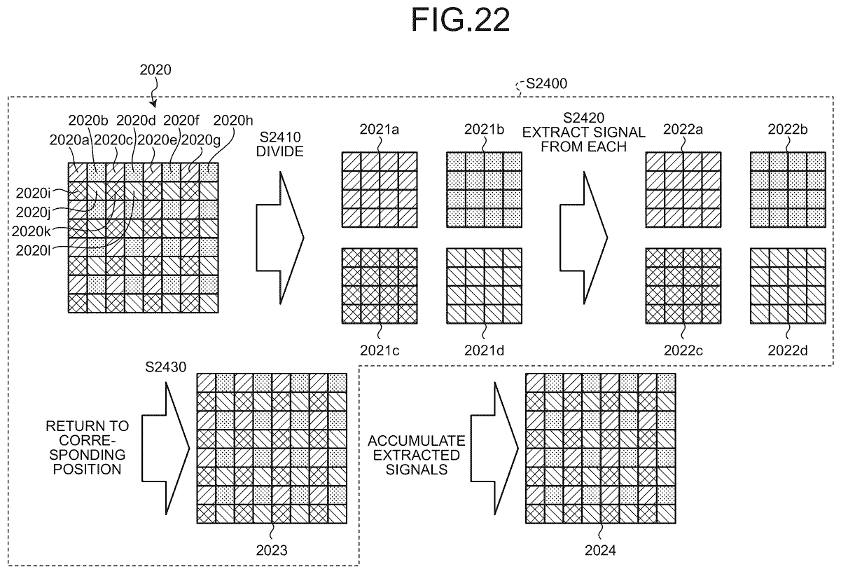

The processing will be explained using FIG. 21 and FIG. 22. FIG. 21 is a diagram explaining about the processing at step S2400 in FIG. 20 in more detail. Moreover, FIG. 22 is a diagram explaining about a flow of the processing at step S2400.

As illustrated in FIG. 22, image data 2020 illustrates the first ultrasonic-image data to which the processing at step S2400 is performed. Pixels 2020a, 2020b, 2020c, 2020d, 2020e, 2020£, 2020g, 2020h, 20201, 2020j, 2020k, and 20201 represent respective pixels of the image data 2020.

First, at step S2410, the processing circuit 110 divides the first ultrasonic-image data into multiple pieces of the second ultrasonic-image data by the dividing function 1110g1. As an example, the processing circuit 110 divides the image data 2020, which is the first ultrasonic-image data, into four pieces of the second ultrasonic-image data by performing data division processing while thinning out at regular intervals by the dividing function 1110g1. As an example, the processing circuit 110 performs the data division processing while skipping every other data point both vertically and horizontally by the dividing function 1110g1 into, for example, image data 2021a including pixels 2020a, 2020c, 2020e, 2020g, and the like, image data 2021b including pixels 2020b, 2020d, 2020f, 2020h, and the like, image data 2021c including pixels 20201, 2020k, and the like, and image data 2021d including pixels 2020j, 20201, and the like as illustrated. That is, the processing circuit 110 performs division of 2×2 by dividing the image data 2020, which is the first ultrasonic-image data, both vertically and horizontally into two parts by the dividing function 1110g1.

Although a case in which the processing circuit 110 performs division of 2×2 by dividing the image data 2020 both vertically and horizontally into two parts by the dividing function 1110g1 has been explained in the example described above, the embodiment is not limited thereto, and the processing circuit 110 may perform division of 3×3 by dividing image data both vertically and horizontally into three parts into 9 pieces of the second ultrasonic-image data by the dividing function 1110g1, or may perform division of 4×4 by dividing image data both vertically and horizontally into four part into 16 pieces of the second ultrasonic-image data. Furthermore, the embodiment is not limited thereto, and the processing circuit 110 may perform division of 2×1 by dividing image data, for example, only vertically into two parts by the dividing function 1110g1 into two pieces of the second ultrasonic-image data, or may perform division of 1×2 by dividing only horizontally into two parts into two pieces of the second ultrasonic-image data.

Subsequently, at step S2420, the processing circuit 110 extracts the third ultrasonic-image data by performing peak extraction from the respective pieces of the second ultrasonic-image data by the extracting function 1110g2.

As an example of the peak detection at step S2420, the processing circuit 110 extracts a high signal point using, for example, a kernel that spans nine signal points of 3×3 including a point of interest and eight signal points adjacent to the point of interest by the extracting function 1110g2. In this case, the processing circuit 110 extracts, only when a point of interest has a higher signal value than any other signal points of the eight adjacent cells, the point of interest as a high signal point by the extracting function 1110g2.

Moreover, as another example of the peak detection processing at step S2420, the processing circuit 110 extracts the third ultrasonic-image data from the second ultrasonic-image data using a kernel that extends in a specific direction, for example, a kernel 4 in a size of 3×1 or the like by the extracting function 1110g2.

Furthermore, as an other example of the peak detection processing at step S2420, the processing circuit 110 extracts the third ultrasonic-image data from the second ultrasonic-image data using a trained model applying a machine learning by the extracting function 1110g2.

As described, the processing circuit 110 extracts the image data 2022a, 2022b, 2022c, and 2022d, which is the third ultrasonic-image data from the image data 2021a, 2021b, 2021c, and 2021d, which is the second ultrasonic-image data, respectively, by the extracting function 1110g2.

Subsequently, at step S2430, the processing circuit 110 generates fourth ultrasonic-image data by allocating the third ultrasonic-image data to a corresponding position on the first ultrasonic-image data by the restoration processing function 1110g3. That is, the processing circuit 110 generates image data 2023, which is the fourth ultrasonic-image data, by allocating the image data 2022a, 2022b, 2022c, and 2022d, which is the third ultrasonic-image data, on the first ultrasonic-image data, by the restoration processing function 1110g3.

Subsquently, at step S2440, the processing circuit 110 generates the fifth ultrasonic-image data by integrating the fourth ultrasonic-image data by the accumulation processing function 1110g4. Subsequently, at step S2450, the processing circuit 110 outputs the fifth ultrasonic-image data by the output function 1110h.

As above, the processing at step S2400 has been explained, and in the embodiment illustrated in FIG. 20, the processing circuit 110 applies the extraction processing described above for each frame by the extracting function 1110g2. That is, the processing circuit 110 applies a kernel to extract a high signal point for each of the multiple frames to extract the fifth ultrasonic-image data by the extracting function 1110g. The processing circuit 110 generates the image data 2024 to be output by summing the fifth ultrasonic-image data in each frame as necessary.

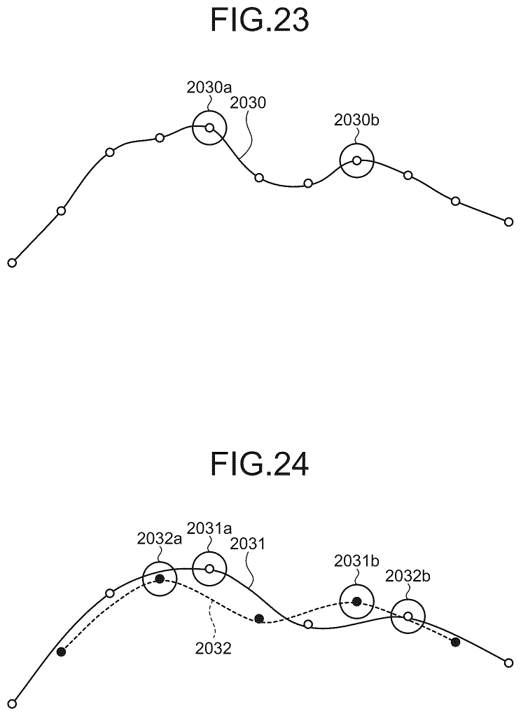

Effects of the high-signal-point extraction processing at step S2400 according to the embodiment will be explained using FIG. 23 and FIG. 24, FIG. 23 is a schematic diagram depicting a high-signal-point extraction method according to a comparative example, while FIG. 24 is a schematic diagram depicting a high-signal-point extraction method according to the embodiment.

FIG. 23 illustrates a high-signal-point extraction method according to the comparative example, and illustrates an extraction method of performing the high-signal point extraction without performing spatial division. Respective points in FIG. 23 represent respective signal points, and a curve 2030 is a line connecting signal values. At step S2420, when the processing circuit 110 performs signal extraction by the extracting function 1110g2, a signal point 30a and a signal point 30b are extracted as a high signal point.

On the other hand, FIG. 24 illustrates the high-signal-point extraction method according to the embodiment, and illustrates an extraction method of performing the high-signal-point extraction by performing spatial division. Respective points in FIG. 24 represent respective signal points, and a curve 2031 and a curve 2032 are lines connecting signal values in the image data in which the division processing is performed at step S2410. When the processing circuit 110 performs, with respect to each of the image data subjected to the division processing at step S2410, signal extraction by the extracting function 1110g2 at step S2420, a signal points 2031a and 2031b, and signal points 2032a and 2032b are extracted as high signal points. That is, by using the method according to the embodiment, a high brightness signal region can be extracted in a relatively wide range compared to the comparative example. That is, in the method of the embodiment, extraction of image components representing a target object is continuous, and a peak pixel value can be extracted continuously, A point of interest for peak determination is basically a pixel unit, but may be a group unit having multiple pixels. When performing peak detection in a group unit, a pixel value for the group may be determined by using an average value of the pixel values included in the multiple groups, or by using an accumulated value.

Referring back to FIG. 20, at step S2500, the processing circuit 110 generates ultrasonic image data, which is composite image data, by performing composition processing of combining between the first ultrasonic-image data generated at step S2300 and the fifth ultrasonic-image data extracted at step S2400 by the output function 1110h. As an example, the processing circuit 110 outputs the composite image data acquired by performing composition processing between the first ultrasonic-image data and the fifth ultrasonic-image data by the output function 1110h.

Specifically, the processing circuit 110 performs weighted addition on the first ultrasonic-image data and the fifth ultrasonic-image data to generate the composite image data by the output function 1110h. For example, the processing circuit 110 sets a weight of the first ultrasonic-image data to 0.8, and sets a weight of the fifth ultrasonic-image data to 0.2, and performs weighted addition on the first ultrasonic-image data and the second ultrasonic-image data, to generate the composite image data by the output function 1110h.

Moreover, at step S2500, the processing circuit 110 may perform composition after normalizing the first ultrasonic-image data and the fifth ultrasonic-image data, to generate the composite image data by the output function 1110h. In other words, the processing circuit 110 may perform composition after performing signal processing on each of signal data such that maximum values or average values of a signal value become equivalent between the first ultrasonic-image data and the fifth ultrasonic-image data, to generate the composite image data by the output function 1110h.

Furthermore, as another example, the processing circuit 110 may generate the correction image data obtained by correcting the first ultrasonic-image data is corrected, by correcting the first ultrasonic-image data using the fifth ultrasonic-image data by the output function 1110h. That is, the processing circuit 110 may output the correction image data that is obtained by subjecting the first ultrasonic-image data to correction processing based on the fifth ultrasonic-image data, which is image components representing a target object successively extracted, by the output function 1110h.

The embodiment is not limited to the example described above, and the processing circuit 110 may additionally perform various kinds of processing on the first ultrasonic-image data or the fifth ultrasonic-image data by the output function 1110h.

As an example, the processing circuit 110 may perform spatial filtering processing such as noise removal, or apply a smoothing filter in a spatial or temporal direction on the fifth ultrasonic-image data generated at step S2400, and may generate the third ultrasonic-image data using signal data on which the spatial filtering processing has been performed or to which the smoothing filter in the spatial direction or the temporal direction has been applied at step S2500, by the output function 1110h. Furthermore, as another example, the processing circuit 110 may generate the ultrasonic-image data using the signal data subjected to the spatial filtering processing at step S2500, after performing processing in the frame direction, for example, operation of replacing signal data with a moving average value of predetermined number of frames, on the fifth ultrasonic-image data generated at step S2400, by the output function 1110h. Moreover, the processing circuit 110 may perform the spatial filtering processing or the processing in the frame direction using AI by the output function 1110h. As an example, the processing circuit 110 may perform the spatial filtering processing on the fifth ultrasonic-image data based on a neural network that has been trained using a low quality image as input data and a high quality image as output data by the output function 1110h.

Furthermore, as another example, these spatial filtering processing and processing in the frame direction may be performed on the generated composite ultrasonic-image data. That is, the processing circuit 110 may perform the spatial filtering processing such as noise removal or the processing in the frame direction, for example, the operation of replacing signal data with a moving average of a predetermined number of frames, on the composite ultrasonic-image data generated at step S2500 by the output function 1110h. Moreover, the processing circuit 110 may perform the spatial filtering processing or the processing in the frame direction on the composite ultrasonic-image data using AI by the output function 1110h.

The processing circuit 110 causes the display 135 to display the generated output ultrasonic-image data by the display control function 114. Moreover, the processing circuit 110 may cause the display 135 to display the fifth ultrasonic-image data by the display control function 114.

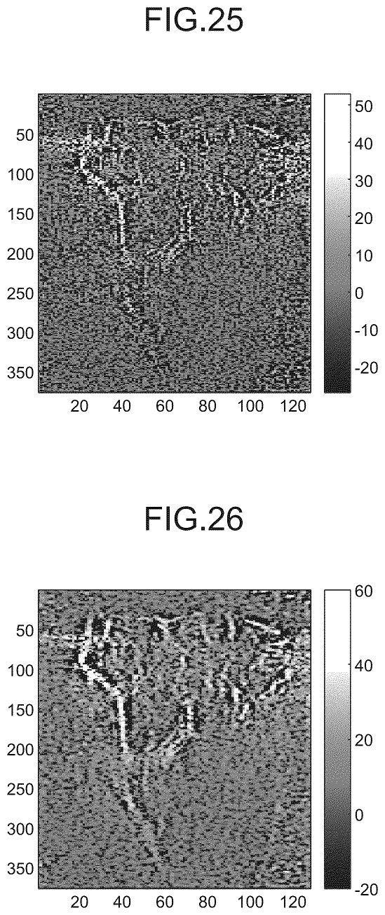

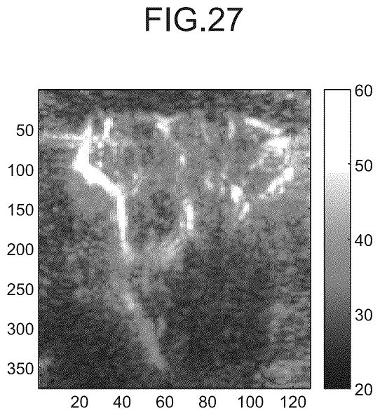

An ultrasonic image 29 output by the method according to the embodiment will be explained using FIGS. 25 to 27. FIG. 25 is a first comparative example, and is an ultrasonic image data acquired by using the super-resolution imaging method explained in FIG. 11. FIG. 26 is an ultrasonic image acquired by using the super-resolution imaging method using the method according to the embodiment. FIG. 27 is a second comparative example, and is an ultrasonic image using the power Doppler method. In the method according to the embodiment, blood flow is rendered smoothly compared to the first comparative example. Moreover, in FIG. 25 and FIG. 26 that are ultrasonic images using the super-resolution imaging method, an image in which blood flow is rendered precisely and at high resolution compared to FIG. 27 that is an ultrasonic image of the conventional blood flow imaging.

In the example in FIG. 20, processing of extracting a high signal point for each frame by the processing circuit 110 through the extraction processing function 1110g at step S2400 has been explained. However, the embodiment is not limited thereto. That is, the processing circuit 110 may perform the kernel application processing at step S2400 with respect to data that has been subjected to the frame addition processing, by the extraction processing function 1110g. A flowchart of the embodiment according to FIG. 28 is illustrated. A flow of processing illustrated in FIG. 28 is the same as that of FIG. 20 except a point that processing at step S2350 is added and, therefore, duplicated explanation about the processing other than step S2350 will be omitted.

At step S2350, the processing circuit 110 acquires addition image data that is obtained by subjecting multiple pieces of frame data consecutive in the temporal direction acquired and collected through ultrasonic scan to addition processing as the first ultrasonic-image data by the acquiring function 110f. At step S2400, the processing circuit 110 performs the extraction processing described above on the first ultrasonic-image data, which is data subjected to the addition processing, and performs the same processing thereafter by the extraction processing function 1110g. Thus, even when the signal intensity of each frame is insufficient, the quality of an output image can be improved by performing frame addition to enhance the signal intensity.

As described above, the image processing apparatus 100 as the medical information processing apparatus according to the embodiment can improve the visibility of a target object, being capable of further enhancing minute blood flow and the like.

The embodiment is not limited to the example described above.

A type of data of the first ultrasonic-image data acquired by the processing circuit 110 using the acquiring function 110f at step S2300 can be various data other than, for example, the Doppler image data. As an example, at step S1300, the processing circuit 110 may acquire B-mode image data as the first ultrasonic-image data by the acquiring function 110f. Because the B-mode image data has a characteristic that a signal value becomes large at an edge portion of an organ, by acquiring the B-mode image data as the first ultrasonic-image data to perform processing, the edge portion of an organ can be rendered in enhanced manner. Furthermore, in the embodiment, a case in which the frame data acquired by the processing circuit 110 using the acquiring function 110f is a blood flow image has been explained, but the embodiment is not limited thereto. The frame data acquired by the processing circuit 110 using the acquiring function 110f may be the reception signal, the measurement signal after DAS, or the blood flow signal after clutter removal. In this case, the processing circuit 110 may acquire one blood flow image that is obtained using addition of multiple frames of the reception image/the measurement image, and the like by the acquiring function 110f as first ultrasonic-image data.

Furthermore, at step S2100, the processing circuit 110 may acquire data for each frequency, instead of data for each frame by the acquiring function 110f. In this case, at step S2200, the processing circuit 110 adds data for each frequency instead of performing the addition processing for a frame, to acquire the first ultrasonic-image data by the acquiring function 110f.

Moreover, the first ultrasonic-image data may be data to which the smoothing filter is applied. That is, the processing circuit 110 may acquire data of multiple frames consecutive in the temporal direction that is acquired based on a result of ultrasonic scan on a subject, to which the smoothing filter is applied in the spatial direction or the temporal direction as the first ultrasonic-image data by the acquiring function 110f.

Furthermore, extraction of a local peak position in the first to the fourth embodiments and the like is not limited to the method described above and may be performed by other methods. For example, by performing peak sharpening that applies a nonlinear function to a signal value in a frame, it may be configured to extract a local peak position. In this case, for example, the extracting function 1110g performs resampling on the first ultrasonic-image data to increase the pixel density and enhance the effective resolution. In the resampling, for example, each pixel may be replaced with multiple smaller pixels. Furthermore, a signal value of each pixel after replacement may be interpolated from an original signal value (signal value before replacement), for example, by bicubic interpolation or the like.

Furthermore, the extracting function 1110g sharpens a local peak by raising each signal value of the resampled first ultrasonic data to a power (for example, to the 8th power, 12th power, and the like). The extracting function 1110g extracts the local peak position, for example, by performing threshold processing or the like on the signal value after being raised to a power. That is, the extracting function 1110g extracts identification information in which a position extracted as the local peak is expressed by “1” and a position not extracted as the local peak is expressed by “O”. The extracting function 1110g extracts identification information from each of the first ultrasonic-image data by performing the sharpening processing of the local peak with respect to multiple pieces of the first ultrasonic-image data, A subject to the sharpening processing of a local peak described above is not limited to the first ultrasonic-image data, and may be frame data also.

Moreover, as for the first embodiment to the fourth embodiment, these may be combined appropriately. As an example, as the method of performing the extraction processing at step S300 in the first embodiment or the second embodiment, the method explained in the third embodiment or the fourth embodiment can be used.

Regarding the embodiments described above, the following notes are disclosed as one aspect and selective features of the invention.

Note: An ultrasound diagnostic apparatus 10 provided in one aspect of the present invention includes a processing circuit 110. The processing circuit 110 acquires first data of multiple frames through scan on a subject, by an acquiring function 110f. The processing circuit 110 extracts second signal data of multiple frames by extracting a signal by a nonlinear processing from the first signal data, by a first extracting function 110g. As an example, the processing circuit 110 extracts the second signal data of multiple frames by extracting a signal, a signal value of which corresponds to a local maximum value or falls within a predetermined range from the first signal data. The processing circuit 110 identifies an inappropriate signal point or an inappropriate frame from the second signal data by an identifying function 110h. The processing circuit 110 generates third signal data based on an identification result of the inappropriate signal point or the inappropriate frame, and the second signal data by a generating function 110e.

Note 2 The in appropriate signal point is a signal point that is estimated not to be a blood flow signal of an observation object.

Note 3 The inappropriate frame is a frame estimated to include a signal point that is not a blood flow signal of an observation object.

Note 4 The processing circuit 110 may generate the third signal data by excluding the inappropriate signal from accumulated signal points, by the generating function 110e. Moreover, the processing circuit 110 may generate the third signal data by accumulating the second signal data while excluding the inappropriate signal point or the inappropriate frame from accumulation target by the generating function 110e.

Note 5 The processing circuit 110 may identify a signal point, the number of signal extraction of which is smaller than a criterion as the inappropriate signal point by the identifying function 110h.

Note 6 The processing circuit 110 may identify a frame in which a change in the number of extracted signals in a frame direction is larger than a criterion as the inappropriate frame by the identifying function 110h.

Note 7 The processing circuit 110 may acquire the first signal data based on signal processing for blood flow imaging by the acquiring function 110f.

Note 8 The processing circuit 110 may identify the inappropriate signal point based on a signal intensity at each point by the identifying function 110h.

Note 9 The processing circuit 110 may identify the inappropriate frame based on the number of extracted signals in the frame direction by the identifying function 110h.

Note 10 The processing circuit 110 may accept an input of the criterion to identify the inappropriate signal point or the inappropriate frame by the identifying function 110h.

Note 11 The processing circuit 110 may display a value of an index value of each position or each frame for a user to set the criterion on a display unit by a display control function 110c.

Note 12 The processing circuit 110 measures the number of times each point at each position is extracted as a high signal point by the identifying function 110h, and generates a table 40 indicating the number of times of signal point extraction at each point. The processing circuit 110 may display information corresponding to the table 40 in which the number of times of signal point extraction at each point is indicated as an index value on a display 135 serving as a display unit by the display control function 110c.

Note 13 The processing circuit 110 may identify the inappropriate signal point or the inappropriate frame using a neural network by the identifying function 110h.

Note 14 A medical image processing apparatus that is an image processing apparatus provided in one aspect of the present invention includes the processing circuit 110. The processing circuit 110 acquires first signal data of multiple frames through scan on a subject by the acquiring function 110f. The processing circuit 110 extracts second signal data of multiple frames by extracting a signal by a nonlinear processing from the first signal data, by the first extracting function 110g. As an example, the processing circuit 110 extracts the second signal data of multiple frames by extracting a signal, a signal value of which corresponds to a local maximum value or falls within a predetermined range from the first signal data by the first extracting function 110g. The processing circuit 110 identifies an inappropriate signal point or an inappropriate frame from the second signal data by the identifying function 110h. The processing circuit 110 generates third signal data based on an identification result of the inappropriate signal point or the inappropriate frame, and the second signal data by a generating function 110e.

Note 15 An ultrasound diagnostic method provided in one aspect of the present invention includes
acquiring first signal data of multiple frames through scan on a subject,
extracting a signal, a signal value of which corresponds to a local maximum value or falls within a predetermined range by a nonlinear processing from the first signal data,
identifying an inappropriate signal point or an inappropriate frame from the second signal data, and
generating third signal data by accumulating the second signal data while excluding the inappropriate signal point or the inappropriate frame from an accumulation target.

According to at least one of the embodiment explained above, it is possible to improve the image quality.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
a processing circuit configured to acquire first signal data of multiple frames through scan on a subject,
extract second signal data of multiple frames by extracting a signal by nonlinear processing from the first signal data,
identify any one of an inappropriate signal point and an inappropriate frame from the second signal data, and
generate third signal data based on an identification result of any one of the inappropriate signal point and the inappropriate frame, and the second signal data.

2. The ultrasound diagnostic apparatus according to claim 1, wherein
the processing circuit is configured to generate the third signal data by excluding the inappropriate signal point from accumulated signal points.

3. The ultrasound diagnostic apparatus according to claim 1, wherein
the processing circuit is configured to generate the third signal data by accumulating the second signal data while excluding any one of the inappropriate signal point and the inappropriate frame from an accumulation target.

4. The ultrasound diagnostic apparatus according to claim 1, wherein
the processing circuit is configured to identify a signal point at a position at which number of signal extraction is smaller than a criterion as the inappropriate signal point.

5. The ultrasound diagnostic apparatus according to claim 1, wherein
the processing circuit is configured to identify a frame in which a change in number of extracted signals in a frame direction is larger than a criterion as the inappropriate frame.

6. The ultrasound diagnostic apparatus according to claim 1, wherein
the processing circuit is configured to acquire the first signal data based on signal processing of blood flow imaging.

7. The ultrasound diagnostic apparatus according to claim 1, wherein
the processing circuit is configured to identify the inappropriate signal point based on a signal intensity at each point.

8. The ultrasound diagnostic apparatus according to claim 1, wherein
the processing circuit is configured to identify the inappropriate frame based on number of extracted signals in a frame direction.

9. The ultrasound diagnostic apparatus according to claim 1, wherein
the processing circuit is configured to accept an input of a criterion to identify any one of the inappropriate signal point and the inappropriate frame, and
cause a display unit to display a value to be an index value for any one of each position and each frame for a user to set the criterion.

10. An image processing apparatus comprising
a processing circuit configured to acquire first signal data of multiple frames through scan on a subject,
extract second signal data of multiple frames by extracting a signal by nonlinear processing from the first signal data,
identify any one of an inappropriate signal point and an inappropriate frame from the second signal data, and
generate third signal data based on an identification result of any one of the inappropriate signal point and the inappropriate frame, and the second signal data.

11. An ultrasound diagnostic method comprising:
acquiring first signal data of multiple frames through scan on a subject;
extracting second signal data of multiple frames by extracting a signal by nonlinear processing from the first signal data;
identifying any one of an inappropriate signal point and an inappropriate frame from the second signal data; and
generating third signal data based on an identification result of any one of the inappropriate signal point and the inappropriate frame, and the second signal data.

12. A medical information processing apparatus comprising
a processing circuit configured to apply a kernel to extract components consecutive in a direction of interest to first ultrasonic image data that is acquired based on a result of ultrasonic scan on a subject, to extract second ultrasonic-image data.

13. The medical information processing apparatus according to claim 12, wherein
the processing circuit is configured to output the second ultrasonic-image data.

14. The medical information processing apparatus according to claim 12, wherein
the processing circuit is configured to extract, when a pixel value of a position of interest is equal to or larger than a pixel value of a point adjacent to the point of interest in one direction, an image component of the point of interest as an image component representing a target object.

15. The medical information processing apparatus according to claim 12, wherein
the processing circuit is configured to perform comparison between a pixel value of the point of interest and a pixel value of an adjacent point for each direction of interest relative to the point of interest, for each of a plurality of points of interest included in the first ultrasonic-image data, and assign a weight to an image component of the point of interest based on a comparison result of each direction of interest.

16. The medical information processing apparatus according to claim 12, wherein
the processing circuit is configured to perform, for each of a plurality of points of interest included in the first ultrasonic-image data, comparison between a pixel value of the point of interest and a pixel value of an adjacent point for each direction of interest relative to the point of interest, and assign a weight to an image component of the point of interest for each direction of interest.

17. The medical information processing apparatus according to claim 12, wherein
the processing circuit is configured to acquire the first ultrasonic-image data based on data of a plurality of frames consecutive in a temporal direction acquired based on a result of ultrasonic scan on a subject, and extract the second ultrasonic-image data by applying a kernel to each of the frames.

18. The medical information processing apparatus according to claim 12, wherein
the processing circuit is configured to acquire data obtained by applying a smoothing filter in any one of a spatial direction and a temporal direction to data of a plurality of frames consecutive in a temporal direction acquired based on a result of ultrasonic scan on a subject, as the first ultrasonic-image data.

19. The medical information processing apparatus according to claim 12, wherein
the processing circuit is configured to collect a plurality of pieces of frame data consecutive in a temporal direction acquired by execution of the ultrasonic scan, and acquire addition image data acquired by performing addition processing on the frame data as the first ultrasonic-image data.

20. An ultrasound diagnostic apparatus comprising a processing circuit configured to cause an ultrasound probe to perform ultrasonic scan on a subject, and extract second ultrasonic-image data by applying a kernel to extract Components consecutive in a direction of interest to first ultrasonic-image data acquired based on a result of the ultrasonic scan.

21. A non-volatile computer-readable recording medium that stores a program causing a computer to execute:
processing of extracting second ultrasonic-image data by applying a kernel to extract components consecutive in a direction interest to first ultrasonic-image data acquired based on a result of ultrasonic scan on a subject.

22. A medical information processing apparatus comprising a processing circuit configured to divide first ultrasonic-image data into a plurality of pieces of second ultrasonic-image data,
extract third ultrasonic-image data by performing peak extraction from each of the second ultrasonic-image data,
generate fourth ultrasonic-image data by allocating the third ultrasonic-image data to a corresponding position on the first ultrasonic-image data, and
generate fifth ultrasonic-image data by integrating the fourth ultrasonic-image data.

23. The medical information processing apparatus according to claim 22, wherein
the processing circuit is configured to output the fifth ultrasonic-image data.

24. The medical information processing apparatus according to claim 22, wherein
the processing circuit further comprises an output unit configured to output composite image data acquired by subjecting the first ultrasonic-image data and the fifth ultrasonic-image data to composition processing.

25. The medical information processing apparatus according to claim 22, wherein
the processing circuit is configured to output correction image data that is acquired by subjecting the first ultrasonic-image data to correction processing based on the fifth ultrasonic-image data.

26. The medical information processing apparatus according to claim 22, wherein
the processing circuit is configured to acquire data of a plurality of frames consecutive in a temporal direction acquired based on a result of ultrasonic scan on a subject as the first ultrasonic-image data.

27. The medical information processing apparatus according to claim 22, wherein
the processing circuit is configured to collect a plurality of pieces of frame data consecutive in a temporal direction acquired through the ultrasonic scan, and acquire addition image data obtained by subjecting the multiple pieces of frame data to addition processing, as the first ultrasonic-image data.

28. An ultrasound diagnostic apparatus comprising
a processing circuit configured to cause an ultrasound probe to perform ultrasonic scan,
divide first ultrasonic-image data into a plurality of second ultrasonic-image data,
perform peak extraction to extract third ultrasonic-image data from each of the second ultrasonic-image data,
generate fourth ultrasonic-image data by allocating the third ultrasonic-image data to a corresponding position on the first ultrasonic-image data, and
generate fifth ultrasonic-image data by integrating the fourth ultrasonic-image data.

29. A non-volatile computer-readable recording medium that stores a program causing a computer to execute:
dividing first ultrasonic-image data into a plurality of pieces of second ultrasonic-image data;
extracting third ultrasonic-image data by performing peak extraction from each of the second ultrasonic-image data;
generating fourth ultrasonic-image data by allocating the third ultrasonic-image data to a corresponding position on the first ultrasonic-image data; and
generating fifth ultrasonic-image data by integrating the fourth ultrasonic-image data.
